(19) United States
(12) Patent Application Publication
(10) Pub. No.: US 2015/0307914 A9
(43) Pub. Date: Oct. 29, 2015

(54) METHODS OF ADMINISTERING MONOMETHYL FUMARATE AND PRODRUGS THEREOF HAVING REDUCED SIDE EFFECTS

(71) Applicant: XenoPort, Inc., Santa Clara, CA (US)

(72) Inventor: Peter A. Virsik, Portola Valley, CA (US)

(21) Appl. No.: 14/449,513

(22) Filed: Aug. 1, 2014

Related U.S. Application Data
(60) Provisional application No. 61/861,256, filed on Aug. 1, 2013.

(51) Int. Cl. C12Q 1/48; A61K 31/225

(52) U.S. Cl. CPC C12Q 1/48; A61K 31/225; G01N 2800/52

(57) ABSTRACT

Methods of improving patient safety and reducing undesirable side effects for patients considering therapeutic treatment using monomethyl fumarate and prodrugs of monomethyl fumarate are disclosed. In particular, a method of treating a disease in a patient in need of such treatment is provided. The method comprises testing the patient for a propensity for a deficiency in tissue glutathione S-transferase theta 1 enzyme (GSTT1) levels. Thereafter, a therapeutically effective amount of a compound selected from monomethyl fumarate (MMF), a prodrug of monomethyl fumarate, and combinations thereof is administered to the patient. During the treatment of the disease, blood lymphocyte concentration is periodically tested in the patient at a predetermined time interval length that is based on the enzyme level propensity testing result.

### CROSS-REFERENCE

This application claims the benefit under 35 U.S.C. §119(e) of U.S. Provisional Application Ser. No. 61/861,256, filed Aug. 1, 2013, and entitled “Methods of Administering Monomethyl Fumarate and Prodrugs Thereof Having Reduced Side Effect,” which is incorporated by reference in its entirety.

### TECHNICAL FIELD

Disclosed herein are methods of increasing patient safety during administration of monomethyl fumarate and/or a prodrug thereof in the treatment of diseases such as multiple sclerosis and psoriasis.

### BACKGROUND

Dimethyl fumarate, a fumaric acid ester (FAE), is approved in Germany for the treatment of psoriasis, and is approved in the United States for the treatment of multiple sclerosis.

FAEs and other fumaric acid derivatives have been proposed for use in treating a wide-variety of diseases and conditions involving immunological, autoimmune, and/or inflammatory processes including psoriasis (Joshi and Strebel, WO 1999/49858 and U.S. Pat. No. 6,277,882; Mrowietz and Asadullah, Trends Mol Med 2005, 111(1), 43-48; and Yazdi and Mrowietz, Clinics Dermatology 2008, 26, 522-526); asthma and chronic obstructive pulmonary diseases (Joshi et al., WO 2005/023241 and US 2007/0027076); cardiac insufficiency including left ventricular insufficiency, myocardial infarction and angina pectoris (Joshi et al., WO 2005/023241; Joshi et al., US 2007/0027076); mitochondrial and neurodegenerative diseases such as Parkinson's disease, Alzheimer's disease, Huntington's disease, retinopathia pigmentosa and mitochondrial encephalomyopathy (Joshi and Strebel, WO 2002/055063, US 2006/0205659, U.S. Pat. No. 6,509,376, U.S. Pat. No. 6,858,750, and U.S. Pat. No. 7,157,423); transplantation (Joshi and Strebel, WO 2002/055063, US 2006/0205659, U.S. Pat. No. 6,359,003, U.S. Pat. No. 6,509,376, and U.S. Pat. No. 7,157,423; and Lehmann et al., Arch Dermatol Res 2002, 294, 399-404); autoimmune diseases (Joshi and Strebel, WO 2002/055063, U.S. Pat. No. 6,509,376, U.S. Pat. No. 7,157,423, and US 2006/0205659) including multiple sclerosis (MS) (Joshi and Strebel, WO 1998/52549 and U.S. Pat. No. 6,436,992; Went and Lieberburg, US 2008/0089896; Schimrigk et al., Eur J Neurology 2006, 13, 604-610; and Schilling et al., Clin Experimental Immunology 2006, 145, 101-107); ischemia and reperfusion injury (Joshi et al., US 2007/0027076); AGE-induced genome damage (Heidland, WO 2005/027899); inflammatory bowel diseases such as Crohn's disease and ulcerative colitis; arthritis; and others (Nilsson et al., WO 2006/037342 and Nilsson and Muller, WO 2007/042034).

Fumaderm®, an enteric coated tablet containing a mixture of salts of monoethyl fumarate and dimethyl fumarate was approved in Germany in 1994 for the treatment of psoriasis. Dimethyl fumarate (DMF) is rapidly metabolized in vivo to monomethyl fumarate (MMF), and hence DMF is considered to be a prodrug of MMF.

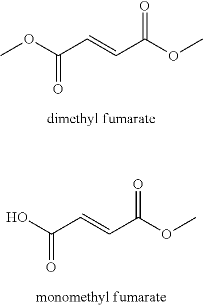

Fumaderm® dosing has been linked to lymphopenia, also referred to as lymphocytopenia, a decrease in lymphocyte counts in a patient's blood. See for example, Antonie et al., Use of Fumaric Acid Esters in Psoriasis, Indian Journal of Dermatology, Venereology and Leprology, Vol. 73, No. 2, March-April, 2007, pp. 133-137.

During the clinical testing of Tecfidera™ (dimethyl fumarate) for the treatment of multiple sclerosis, mean lymphocyte counts decreased in treated patients by approximately 30% during the first year of treatment with the drug, and then remained stable. Four weeks after stopping Tecfidera™, mean lymphocyte counts increased but did not return to baseline. Tecfidera™ also contains the following warnings in its prescribing information; (i) Tecfidera™ may cause lymphopenia; (ii) a recent complete blood count should be available before initiating treatment with Tecfidera™; and (iii) a complete blood count is recommended annually, and as clinically indicated.

### SUMMARY

Disclosed herein are methods of improving patient safety in patients considering being treated with a compound selected from (i) monomethyl fumarate (MMF), (ii) a prodrug of monomethyl fumarate, and (iii) a combination thereof.

In one aspect, there is provided a method of treating a disease in a patient in need of such treatment. The method comprises testing the patient for a propensity for a deficiency in tissue glutathione S-transferase theta 1 enzyme (GSTT1) levels and thereafter administering to the patient a therapeutically effective amount of a compound selected from monomethyl fumarate (MMF), a prodrug of monomethyl fumarate, and combinations thereof. During the treatment, blood lymphocyte concentration in the patient is periodically tested at a predetermined time interval length that is based on the enzyme level propensity testing result.

In another aspect, there is provided a method of treating a disease in a population of patients in need of such treatment, comprising testing each of the patients for a propensity for a deficiency in tissue GSTT1 levels; and thereafter performing one of: (1) administering a compound selected from monomethyl fumarate (MMF), a prodrug of monomethyl fumarate, and combinations thereof only to those patients exhibiting little or no propensity for a deficiency in tissue GSTT1 levels; or (2) administering a compound selected from monomethyl fumarate (MMF), a prodrug of monomethyl fumarate, and combinations thereof to the patients regardless of their enzyme level propensity testing results, and during the treatment, periodically testing blood lymphocyte concentrations in each of the patients at a predetermined time interval length that is based on each patient's own enzyme level propensity testing result.

The enzyme level propensity testing can be either a genotype testing or a phenotype testing. For genotype testing, the testing comprises determining whether the patient exhibits a GSTT1*0/0 genotype, a GSTT1*A/A genotype or a GSTT1*A/0 genotype. For phenotype testing, several possible testing procedures can be used. In one, the testing comprises measuring the amount of S-methyl glutathione formed upon exposure of patient hemoglobin to methyl chloride. In another, the testing comprises measuring the amount of S-methyl glutathione formed upon exposure of patient lymphocytes to methyl chloride.

Regardless of the enzyme testing method used, the time interval length for the subsequent lymphocyte testing is shorter if the enzyme testing shows a propensity for a deficiency in tissue GSTT1 levels and longer if the enzyme testing shows little or no propensity for a deficiency in patient tissue GSTT1 levels. For example, the time interval length is shorter if the patient exhibits a GSTT1*0/0 genotype; and longer if the patient exhibits a GSTT1*A/A genotype or a GSTT1*A/0 genotype. Similar time interval lengths apply for corresponding phenotype testing results.

In a further aspect, the methods include suspending treatment to the patient(s) if the lymphocyte testing shows a lymphocyte blood concentration below about 3,000 cells/μl.

In a further aspect, the methods include suspending treatment to the patient(s) if the lymphocyte testing shows a lymphocyte blood concentration below about 1,500 cells/μl.

The methods are particularly useful in the treatment of diseases such as multiple sclerosis and psoriasis.

In one aspect, the compound being administered comprises monomethyl fumarate.

In another aspect, the compound being administered comprises a prodrug of monomethyl fumarate.

In a third aspect, the prodrug of monomethyl fumarate comprises a compound of Formulae (I), (II), (III), (IV), (V), or (VI) disclosed herein.

Additional embodiments and features are set forth in part in the description that follows, and in part will become apparent to those skilled in the art upon examination of the specification, or may be learned by the practice of the embodiments discussed herein. A further understanding of the nature and advantages of certain embodiments may be realized by reference to the remaining portions of the specification the drawings, the chemical structures, and descriptions, which forms a part of this disclosure. Any description of any R-group or chemical substituent, alone or in any combination, may be used in any chemical Formula described herein, and Formulae include all conformational and stereoisomers, including diastereomers, epimers, and enantiomers.

### BRIEF DESCRIPTION OF THE FIGURES

In addition to the exemplary aspects and embodiments described above, further aspects and embodiments will become apparent by reference to the drawings and by study of the following descriptions.

FIG. 1 is a graph showing GSTT1 genotypes in psoriasis patients and their corresponding GSTT1 enzyme activities (pmol MeSG/mg Hb/min); and

FIG. 2 is a graph showing GSTT1 activity in psoriasis patients, grouped according to non-conjugators (NC), low-conjugators (LC) and high-conjugators (HC).

### DEFINITIONS

A dash (“-”) that is not between two letters or symbols is used to indicate a point of attachment for a moiety or substituent. For example, —CONH₂ is bonded through the carbon atom.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, and alkynyl are used. In certain embodiments, an alkyl group can have from 1 to 20 carbon atoms (C₁₋₂₀) in certain embodiments, from 1 to 10 carbon atoms (C₁₋₁₀), in certain embodiments from 1 to 8 carbon atoms (C₁₋₈), in certain embodiments, from 1 to 6 carbon atoms (C₁₋₆), in certain embodiments from 1 to 4 carbon atoms (C₁₋₄), and in certain embodiments, from 1 to 3 carbon atoms (C₁₋₃).

“Aryl” refers to a monovalent aromatic hydrocarbon radical derived by the removal of one hydrogen atom from a single carbon atom of a parent aromatic ring system. Aryl encompasses benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms chosen from N, O, and S. For such fused, bicyclic ring systems, wherein only one of the rings is a carbocyclic aromatic ring, the radical carbon atom may be at the carbocyclic aromatic ring or at the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), from 6 to 10 carbon atoms (C₆₋₁₀), and in certain embodiments from 6 to 8 carbon atoms (C₆₋₈).

“Arylalkyl” refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀. In certain embodiments, an arylalkyl group is C₇₋₁₂ arylalkyl.

“Compounds” of Formulae (I)-(VI) disclosed herein include any specific compounds within these formulae. Compounds may be identified either by their chemical structure and/or chemical name. Compounds are named using Chemistry 4-D Draw Pro, version 7.01c (ChemInnovation Software, Inc., San Diego, Calif.). When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompasses all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan. Compounds selected from monomethyl fumarate, or a prodrug of monomethyl fumarate such as dimethyl fumarate or a compound of Formulae (I)-(VI), include, but are not limited to, optical isomers thereof, racemates thereof, and other mixtures thereof. In such embodiments, a single enantiomer or diastereomer, i.e., optically active form can be obtained by asymmetric synthesis or by resolution of the racemates. Resolution of the racemates may be accomplished, for example, by conventional methods such as crystallization in the presence of a resolving agent, or chromatography using, for example, chiral stationary phases. Notwithstanding the foregoing, in compounds selected from monomethyl fumarate, or a prodrug of monomethyl fumarate such as dimethyl fumarate or a compound of Formulae (I)-(VI), the configuration of the illustrated double bond is only in the E configuration (i.e. trans configuration).

Compounds selected from monomethyl fumarate, or a prodrug of monomethyl fumarate such as dimethyl fumarate or a compound of Formulae (I)-(VI), may also exist in several tautomeric forms including the enol form, the keto form, and mixtures thereof. Accordingly, the chemical structures depicted herein encompass all possible tautomeric forms of the illustrated compounds. Compounds selected from monomethyl fumarate, or a prodrug of monomethyl fumarate such as dimethyl fumarate or a compound of Formulae (I)-(VI), also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷, etc.

Compounds may exist in unsolvated forms as well as solvated forms, including hydrated forms and as N-oxides. In general, compounds as referred to herein may be free acid, hydrated, solvated, or N-oxides. Certain compounds may exist in multiple crystalline, co-crystalline, or amorphous forms. Compounds selected from monomethyl fumarate, or a prodrug of monomethyl fumarate such as dimethyl fumarate or a compound of Formulae (I)-(VI), include pharmaceutically acceptable salts thereof, or pharmaceutically acceptable solvates of the free acid form of any of the foregoing, as well as crystalline forms of any of the foregoing.

Compounds selected from monomethyl fumarate, or a prodrug of monomethyl fumarate such as dimethyl fumarate or a compound of any of Formulae (I)-(VI), also include solvates. A solvate refers to a molecular complex of a compound with one or more solvent molecules in a stoichiometric or non-stoichiometric amount. Such solvent molecules include those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

Further, when partial structures of the compounds are illustrated, an asterisk (*) indicates the point of attachment of the partial structure to the rest of the molecule.

“Cycloalkyl” refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl.

“Cycloalkylalkyl” refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₁₋₈ and the cycloalkyl moiety is C₃₋₁₂. In certain embodiments, a cycloalkylalkyl group is C₄₋₁₂ cycloalkylalkyl.

“Dimethyl fumarate” refers to the dimethyl ester of fumaric acid. The compound has the formula H₃COOCCH═CHCOOCH₃, and has a molecular weight of 144.13 daltons. This compound is also known by the names Dimethyl (E)-butenedioate (IUPAC), trans-1,2-Ethylenedicarboxylic acid dimethyl ester and (E)-2-Butenedioic acid dimethyl ester. The compound is also referred to herein by the acronym DMF.

“Disease” refers to a disease, disorder, condition, or symptom of any of the foregoing.

“Drug” as defined under 21 U.S.C. §321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals.”

“Halogen” refers to a fluoro, chloro, bromo, or iodo group. In certain embodiments, halogen refers to a chloro group.

“Heteroalkyl” by itself or as part of another substituent refers to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR¹³, ═N—N═, —N═N—, —N═N—NR¹³—, —PR¹³—, —P(O)₂—, —POR¹³—, —O—P(O)₂—, —SO—, —SO₂—, —Sn(R¹³)₂—, and the like, where each R¹³ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹³ is independently chosen from hydrogen and C₁₋₃ alkyl. In certain embodiments, a heteroatomic group is chosen from —O—, —S—, —NH—, —N(CH₃)—, and —SO₂—; and in certain embodiments, the heteroatomic group is —O—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like. In certain embodiments, a heteroaryl group is from 4- to 20-membered heteroaryl (C₄₋₂₀), and in certain embodiments from 4- to 12-membered heteroaryl (C₄₋₁₀). In certain embodiments, heteroaryl groups are those derived from thiophene, pyrrole, benzothiophene, benzofuran, indole, pyridine, quinoline, imidazole, oxazole, or pyrazine. For example, in certain embodiments, C₅ heteroaryl can be furyl, thienyl, pyrrolyl, imidazolyl, pyrazolyl, isothiazolyl, isoxazolyl.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is C₅₋₁₀ heterocycloalkyl, C₅₋₈ heterocycloalkyl, and in certain embodiments, C₅₋₆ heterocycloalkyl.

“Leaving group” has the meaning conventionally associated with it in synthetic organic chemistry, i.e., an atom or a group capable of being displaced by a nucleophile and includes halogen such as chloro, bromo, fluoro, and iodo; acyloxy (alkoxycarbonyl) such as acetoxy and benzoyloxy, aryloxycarbonyl, mesyloxy, tosyloxy and trifluoromethanesulfonyloxy; aryloxy such as 2,4-dinitrophenoxy, methoxy, N, O-dimethylhydroxylamino, p-nitrophenolate, imidazolyl; and the like.

“Lymphopenia”, also sometimes called lymphocytopenia, refers to the condition of having an abnormally low level of lymphocytes in the blood. Lymphocytes are white blood cells with important functions in the immune system. The three types of lymphocytes are B lymphocytes, T lymphocytes, and natural killer cells. Lymphocytes are made in the bone marrow along with other kinds of blood cells. Lymphocytes help protect the body from infection and low lymphocyte concentrations can raise the risk of infection. About 20 to 40 percent of all white blood cells are lymphocytes. Most people who have lymphopenia have low numbers of T lymphocytes. Sometimes they also have low numbers of the other types of lymphocytes. A normal lymphocyte count for adults usually is between 1,000 and 4,800 lymphocytes per microliter of blood. For children, a normal lymphocyte count usually is between 3,000 and 9,500 lymphocytes per microliter of blood. Thus, the term “lymphopenia” typically refers to a count of less than 1,000 to 1,500 lymphocytes per microliter of blood in adults, or less than 3,000 lymphocytes per microliter of blood in children.

Certain factors can cause a low lymphocyte count, such as: the body not making enough lymphocytes; the body making enough lymphocytes, but they're being destroyed; the lymphocytes getting trapped in the spleen or lymph nodes; and/or combinations of any of the above factors. Many diseases, conditions, and factors can cause the above problems that lead to lymphocytopenia. These causes can be acquired (e.g., AIDS) or inherited (e.g., DiGeorge anomaly, Wiskott-Aldrich syndrome, severe combined immunodeficiency syndrome, and ataxia-telangiectasia).

In some cases, lymphopenia can be further classified according to which kind of lymphocytes are reduced. If all three kinds of lymphocytes (T, B and NK lymphocyte types) are suppressed, then the term is used without further qualification. Laboratory tests for determining lymphocyte concentrations (cells/μl) in the blood are well known.

“Monomethyl fumarate” refers to the monomethyl ester of fumaric acid. The compound has the formula HOOCCH═CHCOOCH₃, and has a molecular weight of 130.10 daltons. The compound is also commonly referred to as 2(E)-Butenedioic acid 1-methyl ester, (2E)-4-Methoxy-4-oxobut-2-enoic acid; Fumaric acid hydrogen 1-methyl ester; (2E)-2-Butenedioic acid 1-methyl ester; (E)-2-Butenedioic acid monomethyl ester; Monomethyl trans-ethylene-1,2-dicarboxylate; and methyl hydrogen fumarate. The compound is also referred to herein and elsewhere by the acronyms MMF and/or MHF.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound selected from monomethyl fumarate, or a prodrug of monomethyl fumarate such as dimethyl fumarate or a compound of Formulae (I)-(VI), and at least one pharmaceutically acceptable vehicle, with which the compound is administered to a patient.

“Substituted” refers to a group in which one or more hydrogen atoms are independently replaced with the same or different substituent group(s). In certain embodiments, each substituent group is independently chosen from halogen, —OH, —CN, —CF₃, ═O, —NO₂, benzyl, —C(O)NH₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, —NO₂, benzyl, —R¹¹, —OR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, ═O, —NO₂, benzyl, —C(O)NR¹¹₂, —R¹¹, —OR¹¹, —C(O)R¹¹, COOR¹¹, and —NR¹¹₂ wherein each R¹¹ is independently chosen from hydrogen and C₁₋₄ alkyl. In certain embodiments, each substituent group is independently chosen from —OH, C₁₋₄ alkyl, and —NH₂.

“Systemic administration” and “systemically administering” shall each mean a route of administration of a compound (as defined herein) into the circulatory system of a patient in a therapeutically effective amount (as defined herein). In some non-limiting embodiments, administration can take place via enteral administration (absorption of the medication through the gastrointestinal tract) or parenteral administration (generally injection, infusion, or implantation). These terms are in contrast with topical and other types of local administration where a therapeutically effective amount is not in the circulatory system. In some embodiments, systemic administration is oral administration. In some embodiments, systemic administration is parenteral administration by injection.

“Treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease, or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or is capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

### DETAILED DESCRIPTION

The present disclosure may be understood by reference to the following detailed description, taken in conjunction with the chemical Formulae and definitions as described above. Any description of any R-group or chemical substituent, alone or in any combination, may be used in any chemical Formula described herein, and Formula include all conformational and stereoisomers, including diastereomers, epimers, and enantiomers.

Reference is now made in detail to certain embodiments of methods for increasing patient safety by reducing the incidence of infections during administration of a compound selected from monomethyl fumarate, a prodrug of monomethyl fumarate and combinations thereof. The disclosed embodiments are not intended to be limiting of the claims. To the contrary, the claims are intended to cover all alternatives, modifications, and equivalents.

The methods disclosed herein are useful in increasing patient safety for patients considering treatment with MMF or a prodrug of MMF. Such compounds are known to cause a reduction in lymphocyte concentrations in certain patients, which can lead to an increased incidence rate of opportunistic infections. Thus the methods disclosed herein are effective to prevent such infections by appropriate patient screening and/or testing during MMF or MMF prodrug treatment. The methods disclosed herein are also believed to be useful to prevent opportunistic infections such as multifocal leukoencephalopathy, Kaposi's sarcoma, candidiasis of bronchi, trachea, esophagus, and/or lungs, herpes simplex, bronchitis, pneumonitis, esophagitis and upper respiratory tract infection.

In accordance with the methods disclosed herein, the patient(s) are initially subjected to testing that determines or predicts their propensity for a deficiency in tissue GSTT1 levels. This testing can be either genotype testing or phenotype testing. Suitable genotype test methods are disclosed for example in Sprenger et al. U.S. Pat. No. 6,723,508, the disclosures of which are incorporated herein by reference. Sprenger et al. found that humans typically have one of three GSTT1 genotypes, referred to as GSTT1*A/A genotype, GSTT1*A/0 genotype and GSTT1*0/0 genotype.

It has now been determined that patients in these three genotypes respond differently to dosing with MMF or a prodrug of MMF. Of these three genotypes, the GSTT1*0/0 genotype is the one having the greatest propensity for a deficiency in tissue GSTT1 levels and therefore the highest risk of developing lymphopenia (low white blood cell counts) upon administration of MMF or a prodrug of MMF. The GSTT1*A/A genotype is the one having the lowest propensity for a deficiency in tissue GSTT1 levels and therefore the lowest risk of developing lymphopenia upon administration of MMF or a prodrug of MMF. The GSTT1*A/0 genotype has an intermediate propensity for a deficiency in tissue GSTT1 levels and therefore an intermediate risk of developing lymphopenia upon administration of MMF or a prodrug of MMF. Thus for patients having the GSTT1*A/A genotype, there is a low risk of the patient developing lymphopenia during subsequent administration of MMF or an MMF prodrug and so the subsequent monitoring for low lymphocyte levels is done less frequently, i.e., at longer time intervals. Similarly, for patients having the GSTT1*0/0 genotype, there is a high risk of the patient developing lymphopenia during subsequent administration of MMF or an MMF prodrug and so the subsequent monitoring for low lymphocyte levels is done more frequently, i.e., at shorter time intervals. In addition, for patients having the GSTT1*A/0 genotype, there is an intermediate risk of the patient developing lymphopenia during subsequent administration of MMF or an MMF prodrug and so the subsequent monitoring for low lymphocyte counts can be at frequency that is intermediate the two mentioned above.

For example, for patients having the GSTT1*A/A genotype, the frequency of lymphocyte testing can be every 6 months, every 9 months or even longer. For patients having the GSTT1*A/0 genotype, the frequency of lymphocyte testing can be every 2 to 8 months. For patients having the GSTT1*0/0 genotype, the frequency of lymphocyte testing can be every 1 to 6 months.

Within the above-identified periods of lymphocyte testing frequencies, individual doctors may decide that the frequency of lymphocyte testing can and will vary. In certain embodiments, the lymphocyte testing may occur from 1 day to 7 days, 1 week to 2 weeks, 2 weeks to 3 weeks, 3 weeks to 4 weeks, 1 month to 2 months, 2 months to 3 months, 3 months to 4 months, 4 months to 5 months, 5 months to 6 months, 6 months to 7 months, 7 months to 8 months, 8 months to 9 months, 9 months to 10 months, 10 months to 11 months, or 11 months to 12 months.

Suitable phenotype test methods are disclosed for example in Gambichler et al., Glutathione-S-Transferase T1 Genotyping and Phenotyping in Psoriasis Patients Receiving Treatment with Oral Fumaric Acid Esters, J Eur Acad Derm and Ven, 2013. One of the methods disclosed by Gambichler et al. is measuring the amount of S-methyl glutathione (MeSG) formed upon exposure of patient hemoglobin to methyl chloride. The time interval length for lymphocyte testing is longer if the patient exhibits at least 110 picomoles of S-methyl glutathione formed per minute per ml of hemoglobin (pmol MeSG/mg Hb/min); and shorter if the patient exhibits less than 110 pmol MeSG/mg Hb/min. For example, if the patient exhibits less than 25 pmol MeSG/mg Hb/min then the time interval length for lymphocyte testing is from about 1 to 6 months. If the patient exhibits from about 25 to 110 pmol MeSG/mg Hb/min then the time interval length for lymphocyte testing is from about 2 to 8 months. If the patient exhibits greater than 110 pmol MeSG/mg Hb/min then the time interval length for lymphocyte testing is every 6 months, every 9 months or even longer.

Those skilled in the art will appreciate that a simple variation on the above described phenotype testing comprises measuring the picomole amount of S-methyl glutathione formed per minute per mg of lymphocytes upon exposure of patient lymphocytes to methyl chloride.

Methods and equipment for measuring lymphocyte concentrations in the blood are well known and are considered standard medical laboratory procedures for analyzing human blood. For example, The FACSCanto II™ cytometer (BD Biosciences, San Jose, Calif., USA), equipped with 633 nm and 488 nm red and blue lasers, together with computer hardware and FACSDiva-software™ can used to acquire and analyse lymphocyte populations and subsets. In addition, there are portable devices for measuring patient lymphocyte concentrations in the blood, for example the PortaWBC tester sold by PortaScience Inc. of Moorestown, N.J. Other methods and devices for measuring white blood cell counts are disclosed in Law et al. U.S. Pat. No. 6,709,868.

It is another aspect of the present treatment methods to suspend, either temporarily or permanently, the administration of the MMF or MMF prodrug to any patient(s) whose periodic lymphocyte testing shows lymphocyte concentrations in the blood below about 3000 cells/μl. It is another aspect of the present treatment methods to suspend, either temporarily or permanently, the administration of the MMF or MMF prodrug to any patient(s) whose periodic lymphocyte testing shows lymphocyte concentrations in the blood below about 1500 cells/μl. The MMF or MMF prodrug dosing can be resumed after the patients' lymphocyte concentrations rise back to more acceptable levels, e.g., above 1500 cells/μl and in some cases above 2000 cells/μl.

It is another aspect of the present treatment methods to not administer MMF or an MMF prodrug to any patient that initially exhibits a propensity for a deficiency in tissue GSTT1 levels, for example, in patients exhibiting a GSTT1*0/0 genotype or in patients patient exhibiting less than 25 pmoles of S-methyl glutathione formed per minute per mg of hemoglobin upon exposure of patient hemoglobin to methyl chloride.

In some aspects, the present disclosure provides a method of treating a disease in a patient in need of such treatment. The method comprises testing the patient for a propensity for a deficiency in tissue glutathione S-transferase theta 1 enzyme (GSTT1) levels. Suitable testing methods for a propensity for deficiency in tissue GSTT1 levels are as described above. Thereafter, a therapeutically effective amount of a compound selected from monomethyl fumarate (MMF), a prodrug of monomethyl fumarate, and combinations thereof is administered to the patient. The prodrugs of MMF are as described below. During treatment, the blood lymphocyte concentration may be tested periodically in the patient at a predetermined time interval length that is based on the GSTT1 enzyme level propensity testing result. As used herein, it will be well understood by those in the art that the phrase “during compound administration” means anytime during the course of treating a disease, including during as well as between dosing. The predetermined time interval length may be as described generally herein.

In other aspects of the present disclosure, a method of treating a disease in a population of patients in need of such treatment is provided. The method comprises (a) testing each of the patients for a propensity for a deficiency in tissue glutathione S-transferase theta 1 enzyme (GSTT1) levels; and thereafter performing one of (1) or (2). Suitable testing methods for a propensity for a deficiency in tissue GSTT1 levels are as described above. (1) A compound selected from monomethyl fumarate (MMF), a prodrug of monomethyl fumarate, and combinations thereof is administered only to those patients exhibiting little or no propensity for a deficiency in tissue GSTT1 levels. The MMF prodrugs are as described below. (2) A compound selected from monomethyl fumarate (MMF), a prodrug of monomethyl fumarate, and combinations thereof is administered to the patients regardless of their enzyme level propensity testing results. During treatment, the blood lymphocyte concentration is periodically tested in each of the patients at a predetermined time interval length that is based on each patient's own enzyme level propensity testing result. The predetermined time interval length may be as described generally herein.

In further aspects of the present disclosure, a method of optimizing the safety of treatment of a compound selected from MMF, a prodrug of MMF and combinations thereof, in a patient in need of such treatment is provided. That is, the method is used to optimize the safety of using a compound to treat a patient in need thereof, wherein the compound is selected from MMF, a prodrug of MMF, and combinations thereof. The MMF prodrug is as described below. The method comprises testing the patient for a propensity for a deficiency in tissue glutathione S-transferase theta 1 enzyme (GSTT1) levels. Suitable testing methods for a propensity for a deficiency in tissue GSTT1 levels are as described above. Once testing for propensity for a deficiency in tissue GSTT1 levels has occurred, then a therapeutically effective amount of the compound is administered to the patient, and blood lymphocyte concentration is periodically tested in the patient at a predetermined time interval length that is based on the enzyme level propensity testing result. The predetermined time interval length may be as described generally herein.

### MMF Prodrug Compounds

Certain embodiments of the methods disclosed herein utilize a compound of Formula (I):

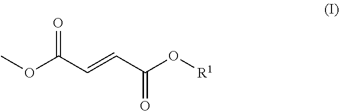

or a pharmaceutically acceptable salt thereof, wherein R¹ is chosen from a C₁ to C₆ alkyl.

In certain embodiments, R¹ is C₂ to C₆ alkyl.

In certain embodiments, R¹ is methyl.

In certain embodiments, R¹ is ethyl, n-propyl, isopropyl, n-butyl, isobutyl, tert-butyl, n-pentyl, pentyl-2-yl, 2-methylbutyl, isopentyl, 3-methylbutan-2-yl, neopentyl, tert-pentyl, n-hexyl, hexan-2-yl, 2-methylpentyl, 3-methylpentyl, 4-methylpentyl, 3-methylpentan-2-yl, 4-methylpentan-2-yl, 2,3-dimethylbutyl, or 3,3-dimethylbutyl.

Examples of compounds of Formula (I) include dimethylfumarate, diethylfumarate, dipropylfumarate, dibutylfumarate, dipentylfumarate, methyl-ethylfumarate, methyl-propylfumarate, methyl-butylfumarate, methyl-pentylfumarate, monoethylfumarate, monopropylfumarate, monobutylfumarate and monopentylfumarate, and/or pharmaceutically acceptable salts of any of the foregoing. In certain embodiments, the compounds of Formula (I) include dimethyl fumarate, methyl ethyl fumarate, methyl n-propyl fumarate and methyl i-propyl fumarate, including pharmaceutically acceptable salts thereof. Pharmaceutically acceptable salts thereof comprise metal salts, such as a salt selected from alkali metal salts and alkaline earth metal salts including sodium, potassium, calcium, magnesium, strontium or zinc salts, amino acid salts etc.

Certain embodiments of the methods disclosed herein utilize a compound of Formula (II):

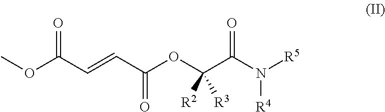

or a pharmaceutically acceptable salt thereof, wherein:

R² and R³ are independently chosen from hydrogen, C₁₋₆ alkyl, and substituted C₁₋₆ alkyl;

R⁴ and R⁵ are independently chosen from hydrogen, C₁₋₆ alkyl, substituted C₁₋₆ alkyl, C₁₋₆ alkoxy, substituted C₁₋₆ alkoxy, C₁₋₆ alkoxycarbonyl, substituted C₁₋₆ alkoxycarbonyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R⁴ and R⁵ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl ring; and

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In certain embodiments of a compound of Formula (II), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl. In certain embodiments, each substituent group is independently chosen from —OH, and —COOH.

In certain embodiments of a compound of Formula (II), each substituent group is independently chosen from ═O, C₁₋₄ alkyl, and —COOR¹¹ wherein R¹¹ is chosen from hydrogen and C₁₋₄ alkyl.

In certain embodiments of a compound of Formula (II), each of R² and R³ is hydrogen.

In certain embodiments of a compound of Formula (II), one of R² and R³ is hydrogen and the other of R² and R³ is C₁₋₄ alkyl.

In certain embodiments of a compound of Formula (II), one of R² and R³ is hydrogen and the other of R² and R³ is chosen from methyl, ethyl, n-propyl, isopropyl, n-butyl, isobutyl, sec-butyl, and tert-butyl.

In certain embodiments of a compound of Formula (II), one of R² and R³ is hydrogen and the other of R² and R³ is methyl.

In certain embodiments of a compound of Formula (II), R⁴ and R⁵ are independently chosen from hydrogen and C₁₋₆ alkyl.

In certain embodiments of a compound of Formula (II), R⁴ and R⁵ are independently chosen from hydrogen and C₁₋₄ alkyl.

In certain embodiments of a compound of Formula (II), R⁴ and R⁵ are independently chosen from hydrogen, methyl, and ethyl.

In certain embodiments of a compound of Formula (II), each of R⁴ and R⁵ is hydrogen; in certain embodiments, each of R⁴ and R⁵ is methyl; and in certain embodiments, each of R⁴ and R⁵ is ethyl.

In certain embodiments of a compound of Formula (II), R⁴ is hydrogen; and R⁵ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl.

In certain embodiments of a compound of Formula (II), R⁴ is hydrogen; and R⁵ is chosen from C₁₋₄ alkyl, benzyl, 2-methoxyethyl, carboxymethyl, carboxypropyl, 1,3,4-thiadiazolyl, methoxy, 2-methoxycarbonyl, 2-oxo(1,3-oxazolidinyl), 2-(methylethoxy)ethyl, 2-ethoxyethyl, (tert-butyloxycarbonyl)methyl, (ethoxycarbonyl)methyl, carboxymethyl, (methylethyl)oxycarbonylmethyl, and ethoxycarbonylmethyl.

In certain embodiments of a compound of Formula (II), R⁴ and R⁵ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (II), R⁴ and R⁵ together with the nitrogen to which they are bonded form a ring chosen from a C₅ heterocycloalkyl, substituted C₅ heterocycloalkyl, C₅ heteroaryl, and substituted C₅ heteroaryl ring. In certain embodiments of a compound of Formula (II), R⁴ and R⁵ together with the nitrogen to which they are bonded form a ring chosen from a C₆ heterocycloalkyl, substituted C₆ heterocycloalkyl, C₆ heteroaryl, and substituted C₆ heteroaryl ring. In certain embodiments of a compound of Formula (II), R⁴ and R⁵ together with the nitrogen to which they are bonded form a ring chosen from piperazine, 1,3-oxazolidinyl, pyrrolidine, and morpholine ring.

In certain embodiments of a compound of Formula (II), one of R² and R³ is hydrogen and the other of R² and R³ is C₁₋₆ alkyl; R⁴ is hydrogen; and R⁵ is chosen from hydrogen, C₁₋₆ alkyl, and benzyl.

In certain embodiments of a compound of Formula (II), one of R² and R³ is hydrogen and the other of R² and R³ is C₁₋₆ alkyl; R⁴ is methyl; and R⁵ is chosen from hydrogen, C₁₋₆ alkyl, and benzyl.

In certain embodiments of a compound of Formula (II), one of R² and R³ is hydrogen and the other of R² and R³ is chosen from hydrogen and C₁₋₆ alkyl; and each of R⁴ and R⁵ is C₁₋₆ alkyl.

In certain embodiments of a compound of Formula (II), one of R² and R³ is hydrogen and the other of R² and R³ is chosen from hydrogen and C₁₋₆ alkyl; and each of R⁴ and R⁵ is C₁₋₆ alkyl. In certain embodiments of a compound of Formula (II), each of R² and R³ is hydrogen; and each of R⁴ and R⁵ is C₁₋₆ alkyl.

In certain embodiments of a compound of Formula (II), one of R² and R³ is hydrogen and the other of R² and R³ is chosen from hydrogen and C₁₋₄ alkyl; R⁴ is hydrogen; and R⁵ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl. In certain embodiments of a compound of Formula (II), one of R² and R³ is hydrogen and the other of R² and R³ is methyl; R⁴ is hydrogen; and R⁵ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl. In certain embodiments of a compound of Formula (II), each of R² and R³ is hydrogen; R⁴ is hydrogen; and R⁵ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl.

In certain embodiments of a compound of Formula (II), R⁴ and R⁵ together with the nitrogen to which they are bonded form a C₅₋₁₀ heterocycloalkyl ring.

In certain embodiments of a compound of Formula (II), one of R² and R³ is hydrogen and the other of R² and R³ is chosen from hydrogen and C₁₋₆ alkyl; and R⁴ and R⁵ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (II), one of R² and R³ is hydrogen and the other of R² and R³ is methyl; and R⁴ and R⁵ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (II), each of R² and R³ is hydrogen; and R⁴ and R⁵ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring.

In certain embodiments of a compound of Formula (II), one of R² and R³ is hydrogen and the other of R² and R³ is chosen from hydrogen and C₁₋₆ alkyl; and R⁴ and R⁵ together with the nitrogen to which they are bonded form a ring chosen from morpholine, piperazine, and N-substituted piperazine.

In certain embodiments of a compound of Formula (II), one of R² and R³ is hydrogen and the other of R² and R³ is chosen from hydrogen and C₁₋₆ alkyl; and R⁴ and R⁵ together with the nitrogen to which they are bonded form a ring chosen from morpholine, piperazine, and N-substituted piperazine.

In certain embodiments of a compound of Formula (II), R² is hydrogen, and in certain embodiments, R³ is hydrogen.

In certain embodiments of a compound of Formula (II), R⁴ and R⁵ are independently chosen from hydrogen, C₁₋₆ alkyl, substituted C₁₋₆ alkyl, C₆₋₁₀ aryl, substituted C₆₋₁₀ aryl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₆₋₁₀ heteroaryl, substituted C₆₋₁₀ heteroaryl, C₄₋₁₂ heterocycloalkylalkyl, substituted C₄₋₁₂ heterocycloalkylalkyl, C₇₋₁₂ heteroarylalkyl, substituted C₇₋₁₂ heteroarylalkyl; or R⁴ and R⁵ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl.

In certain embodiments, the Formula (II) compound is chosen from:
(N,N-diethylcarbamoyl)methyl methyl (2E)but-2-ene-1,4-dioate;
methyl [N-benzylcarbamoyl]methyl (2E)but-2-ene-1,4-dioate;
methyl 2-morpholin-4-yl-2-oxoethyl (2E)but-2-ene-1,4-dioate;
(N-butylcarbamoyl)methyl methyl (2E)but-2-ene-1,4-dioate;
[N-(2-methoxyethyl)carbamoyl]methyl methyl (2E)but-2-ene-1,4-dioate;
2-{2-[(2E)-3-(methoxycarbonyl)prop-2-enoyloxy]acetylamino}acetic acid;
4-{2-[(2E)-3-(methoxycarbonyl)prop-2-enoyloxy]acetylamino}butanoic acid;
methyl (N-(1,3,4-thiadiazol-2-yl)carbamoyl)methyl (2E)but-2-ene-1,4-dioate;
(N,N-dimethylcarbamoyl)methyl methyl (2E)but-2-ene-1,4-dioate;
(N-methoxy-N-methylcarbamoyl)methyl methyl (2E)but-2-ene-1,4-dioate;
bis-(2-methoxyethylamino)carbamoyl]methyl methyl (2E)but-2-ene-1,4-dioate;
[N-(methoxycarbonyl)carbamoyl]methyl methyl (2E)but-2ene-1,4-dioate;
4-{2-[(2E)-3-(methoxycarbonyl)prop-2-enoyloxy]acetylamino}butanoic acid, sodium salt;
methyl 2-oxo-2-piperazinylethyl (2E)but-2-ene-1,4-dioate;
methyl 2-oxo-2-(2-oxo(1,3-oxazolidin-3-yl)ethyl (2E)but-2-ene-1,4-dioate;
{N-[2-(dimethylamino)ethyl]carbamoyl}methyl methyl (2E)but-2-ene-1,4 dioate;
methyl 2-(4-methylpiperazinyl)-2-oxoethyl (2E)but-2-ene-1,4-dioate;
methyl {N-[(propylamino)carbonyl]carbamoyl}methyl (2E)but-2-ene-1,4-dioate;
2-(4-acetylpiperazinyl)-2-oxoethyl methyl (2E)but-2-ene-1,4-dioate;
{N,N-bis[2-(methylethoxy)ethyl]carbamoyl}methyl methyl (2E)but-2-ene-1,4-dioate;
methyl 2-(4-benzylpiperazinyl)-2-oxoethyl (2E)but-2-ene-1.4-dioate;
[N,N-bis(2-ethoxyethyl)carbamoyl]methyl methyl (2E)but-2-ene-1,4-dioate;
2-{(2S)-2-[(tert-butyl)oxycarbonyl]pyrrolidinyl}-2-oxoethyl methyl (2E)but-2ene-1,4-dioate;
1-{2-{(2E)-3-(methoxycarbonyl)prop-2-enoyloxy]acetyl}(2S)pyrrolidine-2-carboxylic acid;
(N-{[tert-butyl)oxycarbonyl]methyl}-N-methylcarbamoyl)methyl methyl (2E)but-2-ene-1,4-dioate;
{N-(ethoxycarbonyl)methyl]-N-methylcarbamoyl}methyl methyl (2E)but-2-ene-1,4-dioate;
methyl 1-methyl-2-morpholin-4-yl-2-oxoethyl (2E)but-2-ene-1,4-dioate;
(1S)-[N,N-bis(2-methoxyethyl)carbamoyl]ethyl methyl (2E)but-2-ene-1,4-dioate;
(1S)—(N,N-dimethylcarbamoyl)ethyl methyl (2E)but-2-ene-1,4-dioate;
2-{2-[(2E)-3-(methoxycarbonyl)prop-2-enoyloxyl]-N-methylacetylamino}acetic acid;
(N-{[(tert-butyl)oxycarbonyl]methyl}carbamoyl)methyl methyl (2E)but-2-ene-1,4-dioate;
methyl (N-methyl-N-{[(methylethyl)oxycarbonyl]methyl}carbamoyl)methyl (2E)but-2-ene-1,4-dioate;
{N-[(ethoxycarbonyl)methyl]-N-benzylcarbamoyl}methyl methyl (2E)but-2-ene-1,4-dioate;
1-{N-[(ethoxycarbonyl)methyl]-N-benzylcarbamoyl}ethyl methyl (2E)but-2-ene-1,4-dioate;
1-{N-[(ethoxycarbonyl)methyl]-N-methylcarbamoyl}ethyl methyl (2E)but-2-ene-1,4-dioate;
(1S)-1-methyl-2-morpholin-4-yl-2-oxoethyl methyl (2E)but-2-ene-1,4-dioate;
(1S)-1-[N,N-bis(2-methoxyethyl)carbamoyl]ethyl methyl (2E)but-2-ene-1,4-dioate;
(1R)-1-(N,N-diethylcarbamoyl)ethyl methyl (2E)but-2-ene-1,4-dioate; and

pharmaceutically acceptable salts of any of the foregoing.

In certain embodiments of a compound of Formula (II), the compound is chosen from:
(N,N-diethylcarbamoyl)methyl methyl (2E)but-2-ene-1,4-dioate;
methyl [N-benzylcarbamoyl]methyl (2E)but-2-ene-1,4-dioate;
methyl 2-morpholin-4-yl-2-oxoethyl (2E)but-2-ene-1,4-dioate;
(N-butylcarbamoyl)methyl methyl (2E)but-2-ene-1,4-dioate;
[N-(2-methoxyethyl)carbamoyl]methyl methyl (2E)but-2-ene-1,4-dioate;
2-{2-[(2E)-3-(methoxycarbonyl)prop-2-enoyloxy]acetylamino}acetic acid;
{2-[(2E)-3-(methoxycarbonyl)prop-2-enoyloxy]acetylamino}butanoic acid;
Methyl (N-(1,3,4-thiadiazol-2yl)carbamoyl)methyl (2E)but-2ene-1,4-dioate;
(N,N-dimethylcarbamoyl)methyl methyl (2E)but-2-ene-1,4-dioate;
(N-methoxy-N-methylcarbamoyl)methyl methyl (2E)but-2-ene-1,4-dioate;
bis-(2-methoxyethylamino)carbamoyl]methyl methyl (2E)but-2-ene-1,4-dioate;
[N-(methoxycarbonyl)carbamoyl]methyl methyl (2E)but-2ene-1,4-dioate;
methyl 2-oxo-2-piperazinylethyl (2E)but-2-ene-1,4-dioate;
methyl 2-oxo-2-(2-oxo(1,3-oxazolidin-3yl)ethyl (2E)but-2ene-1,4-dioate;
{N-[2-(dimethylamino)ethyl]carbamoyl}methyl methyl (2E)but-2ene-1,4 dioate;
(N-[(methoxycarbonyl)ethyl]carbamoyl)methyl methyl (2E)but-2-ene-1,4-dioate;
2-{2-[(2E)-3-(methoxycarbonyl)prop-2-enoyloxy]acetylamino}propanoic acid; and

pharmaceutically acceptable salts of any of the foregoing.

In certain embodiments of a compound of Formula (II), the compound is selected from (N,N-diethylcarbamoyl)methyl methyl (2E)but-2-ene-1,4-dioate:

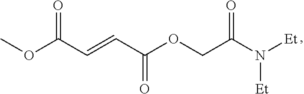 [SMILES: CCN(CC)C(=O)COC(=O)/C=C/C(=O)OC]

or a pharmaceutically acceptable salt thereof;

and (methyl 2-morpholin-4-yl-2-oxoethyl (2E)but-2-ene-1,4-dioate:

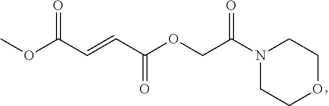 [SMILES: COC(=O)/C=C/C(=O)OCC(=O)N1CCOCC1]

or a pharmaceutically acceptable salt thereof.

Certain embodiments of the methods disclosed herein utilize a compound of Formula (III):

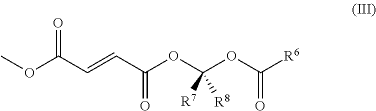

or a pharmaceutically acceptable salt thereof, wherein:

R⁶ is chosen from C₁₋₆ alkyl, substituted C₁₋₆ alkyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl; and

R⁷ and R⁸ are independently chosen from hydrogen, C₁₋₆ alkyl, and substituted C₁₋₆ alkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, N(R¹¹)C(O)C(R¹¹)₂N¹¹₂, and —NR¹¹₂ wherein each R¹¹ is independently chosen from hydrogen and C₁₋₄ alkyl.

In certain embodiments of a compound of Formula (III), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl.

In certain embodiments of a compound of Formula (III), each substituent group is independently chosen from ═O, C₁₋₄ alkyl, and —COOR¹¹ wherein R¹¹ is chosen from hydrogen and C₁₋₄ alkyl.

In certain embodiments of a compound of Formula (III), one of R⁷ and R⁸ is hydrogen and the other of R⁷ and R⁸ is C₁₋₆ alkyl. In certain embodiments of a compound of Formula (III), one of R⁷ and R⁸ is hydrogen and the other of R⁷ and R⁸ is C₁₋₄ alkyl.

In certain embodiments of a compound of Formula (III), one of R⁷ and R⁸ is hydrogen and the other of R⁷ and R⁸ is chosen from methyl, ethyl, n-propyl, and isopropyl. In certain embodiments of a compound of Formula (III), each of R⁷ and R⁸ is hydrogen.

In certain embodiments of a compound of Formula (III), R⁶ is C₁₋₆ alkyl; one of R⁷ and R⁸ is hydrogen and the other of R⁷ and R⁸ is C₁₋₆ alkyl.

In certain embodiments of a compound of Formula (III), R⁶ is —OR¹⁰.

In certain embodiments of a compound of Formula (III), R¹⁰ is chosen from C₁₋₄ alkyl, cyclohexyl, and phenyl.

In certain embodiments of a compound of Formula (III), R⁶ is chosen from methyl, ethyl, n-propyl, and isopropyl; one of R⁷ and R⁸ is hydrogen and the other of R⁷ and R⁸ is chosen from methyl, ethyl, n-propyl, and isopropyl.

In certain embodiments of a compound of Formula (III), R⁶ is substituted C₁₋₂ alkyl, wherein each of the one or more substituent groups are chosen from —COOH, —NHC(O)CH₂NH₂, and —NH₂.

In certain embodiments of a compound of Formula (III), R⁶ is chosen from ethoxy, methylethoxy, isopropyl, phenyl, cyclohexyl, cyclohexyloxy, —CH(NH₂)CH₂COOH, —CH₂CH(NH₂)COOH, —CH(NHC(O)CH₂NH₂)—CH₂COOH, and —CH₂CH(NHC(O)CH₂NH₂)—COOH.

In certain embodiments of a compound of Formula (III), one of R⁷ and R⁸ is hydrogen and the other of R⁷ and R⁸ is chosen from hydrogen, methyl, ethyl, n-propyl, and isopropyl; and R⁶ is chosen from C₁₋₃ alkyl and substituted C₁₋₃ alkyl, wherein each of the one or more substituent groups are chosen from —COOH, —NHC(O)CH₂NH₂, and —NH₂, —OR¹⁰ wherein R¹⁰ is chosen from C₁₋₃ alkyl and cyclohexyl, phenyl, and cyclohexyl.

In certain embodiments of a compound of Formula (III), the compound is chosen from:
[1-(ethoxycarbonyloxy)]ethyl methyl (2E)but-2-ene-1,4-dioate;
methyl [1-(methylethoxycarbonyloxy)]ethyl (2E)but-2-ene-1,4-dioate;
[1-(cyclohexyloxycarbonyloxy)]ethyl methyl (2E)but-2-ene-1,4-dioate; and

pharmaceutically acceptable salts of any of the foregoing.

In certain embodiments of a compound of Formula (III), the compound is chosen from:
methyl (2-methylpropanoyloxy)ethyl (2E)but-2-ene-1,4-dioate;
methyl [1-(phenylcarbonyloxy)]ethyl (2E)but-2-ene-1,4-dioate;
[1-(cyclohexylcarbonyloxy)]butyl methyl (2E)but-2-ene-1,4-dioate;
1-[(2E)-3-(methoxycarbonyl)prop-2-enoyloxy]ethyl methyl (2E)but-2-ene-1,4-dioate;
methyl 2-methyl-1-phenylcarbonyloxypropyl (2E)but-2-ene-1,4-dioate; and

pharmaceutically acceptable salts of any of the foregoing.

In certain embodiments of a compound of Formula (III), the compound is chosen from:
[1-(ethoxycarbonyloxy)]ethyl methyl (2E)but-2-ene-1,4-dioate;
methyl [1-(methylethoxycarbonyloxy)]ethyl (2E)but-2-ene-1,4-dioate;
methyl [1-(2-methylpropanoyloxy)]ethyl (2E)but-2-ene-1,4-dioate;
methyl[1-phenylcarbonyloxy]ethyl (2E)but-2-ene-1,4-dioate;
[1-cyclohexylcarbonyloxy]butyl methyl (2E)but-2-ene-1,4-dioate;
[(2E)-3-(methoxycarbonyl)prop-2-enoyloxy]ethyl methyl (2E)but-2-ene-1,4-dioate;
[1-(cyclohexyloxycarbonyloxy)]ethyl methyl (2E)but-2-ene-1,4-dioate;
methyl 2-methyl-1-phenylcarbonyloxypropyl (2E)but-2-ene-1,4-dioate;
3-({[(2E)-3-(methoxycarbonyl)prop-2-enoyloxy]methyl}oxycarbonyl)(3S)-3-aminopropanoic acid;
3-({[(2E)-3-(methoxycarbonyl)prop-2-enoyloxy]methyl}oxycarbonyl)(2S)-2-aminopropanoic acid;
3-({[(2E)-3-(methoxycarbonyl)prop-2-enoyloxy]methyl}oxycarbonyl)(3S)-3-(2-aminoacetylamino)propanoic acid;
3-({[(2E)-3-(methoxycarbonyl)prop-2-enoyloxy]methyl}oxycarbonyl)(2S)-(2-aminoacetylamino)propanoic acid;

and

pharmaceutically acceptable salts of any of the foregoing.

In certain embodiments of a compound of Formula (III), the compound is chosen from:
3-({[(2E)-3-(methoxycarbonyl)prop-2-enoyloxy]methyl}oxycarbonyl)(3S)-3-aminopropanoic acid, 2,2,2-trifluoroacetic acid salt;
3-({[(2E)-3-(methoxycarbonyl)prop-2-enoyloxy]methyl}oxycarbonyl)(2S)-2-aminopropanoic acid, 2,2,2-trifluoroacetic acid salt;
3-({[(2E)-3-(methoxycarbonyl)prop-2-enoyloxy]methyl}oxycarbonyl)(3S)-3-(2-aminoacetylamino)propanoic acid, 2,2,2-trifluoroacetic acid salt;
3-({[(2E)-3-(methoxycarbonyl)prop-2-enoyloxy]methyl}oxycarbonyl)(2S)-(2-aminoacetylamino)propanoic acid, 2,2,2-trifluoroacetic acid salt;
3-{{1-{[(2E)-3-(methoxycarbonyl)prop-2-enoyloxy]}ethoxycarbonyl}} (2S)-2-aminopropanoic acid, hydrochloride salt; and

pharmaceutically acceptable salts of any of the foregoing.

Certain embodiments of the methods disclosed herein utilize a compound of Formula (IV):

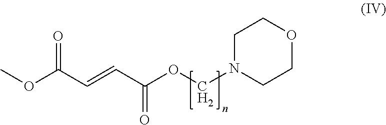

or a pharmaceutically acceptable salt thereof, wherein n is an integer from 2 to 6.

In certain embodiments of a compound of Formula (IV), n is 2, n is 3, n is 4, n is 5, and in certain embodiments, n is 6.

In certain embodiments of a compound of Formula (IV), the compound is chosen from:
Methyl 3-morpholin-4-ylpropyl (2E)but-2-ene-1,4-dioate;
Methyl 4-morpholin-4-ylbutyl (2E)but-2-ene-1,4-dioate;
Methyl 5-morpholin-4-ylpentyl (2E)but-2-ene-1,4-dioate, and pharmaceutically acceptable salts thereof.

Certain embodiments of the methods disclosed herein utilize a compound of Formula (V), similar to, or the same as, those described in Chao, US Patent Application Publication No. 2014/0194427 at paragraphs 33-48, 50 and 51, the disclosure of which is incorporated herein by reference. Compounds of Formula (V) include:

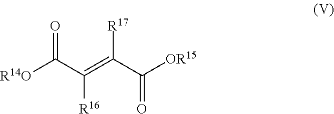

or a pharmaceutically acceptable salt thereof, wherein

each of R¹⁴ and R¹⁵, independently, is selected from the group consisting of hydrogen, deuterium, deuterated methyl, deuterated ethyl, C₁₋₆ alkyl, phenyl, 3-7 membered saturated or partially unsaturated monocyclic cycloalkyl ring, 3-7 membered saturated or partially unsaturated monocyclic heterocyclic ring having 1-3 heteroatoms independently selected from nitrogen, oxygen, and sulfur, and a 5-6 membered heteroaryl ring having 1-3 heteroatoms independently selected from nitrogen, oxygen, and sulfur; and

each of R¹⁶ and R¹⁷, independently, is hydrogen or deuterium.

In certain embodiments of a compound of Formula (V), each of R¹⁴ and R¹⁵, independently, is selected from the group consisting of hydrogen, deuterium, deuterated methyl, deuterated ethyl, and C₁₋₆ alkyl; and

each of R¹⁶ and R¹⁷ independently, is hydrogen or deuterium.

In certain embodiments of a compound of Formula (V), R¹⁴ is hydrogen or —CH₃. In certain embodiments of a compound of Formula (V), R¹⁴ is —CD₃. In certain embodiments of a compound of Formula (V), R¹⁴ is —CD₂CD₃.

In certain embodiments of a compound of Formula (V), R¹⁵ is —CH₂D, —CHD₂, or —CD₃. In certain embodiments of a compound of Formula (V), R¹⁵ is H, —CH₃, —CH₂D, —CHD₂, or —CD₃.

In certain embodiments of a compound of Formula (V), R¹⁴ is hydrogen or —CH₃ and R¹⁵ is —CH₂D, —CHD₂, or —CD₃.

In certain embodiments of a compound of Formula (V), R¹⁴ is —CD₃ and R¹⁵ is —CH₂D, —CHD₂, or —CD₃.

In certain embodiments of a compound of Formula (V), at least one of R¹⁶ and R¹⁷ is deuterium. In certain embodiments of a compound of Formula (V), both of R¹⁶ and R¹⁷ are deuterium.

In certain embodiments of a compound of Formula (V), at least one of R¹⁶ and R¹⁷ is deuterium and R¹⁵ is hydrogen, —CH₃, —CH₂D, —CHD₂, or —CD₃. In certain embodiments of a compound of Formula (V), both of R¹⁶ and R¹⁷ are deuterium and R¹⁵ is hydrogen, —CH₃, —CH₂D, —CHD₂, or —CD₃.

In certain embodiments of a compound of Formula (V), R¹⁴ is —CD₂CD₃ and R¹⁵ is H, —CH₃, —CH₂D, —CHD₂, or —CD₃.

In certain embodiments, the compound of Formula (V) is selected from the group consisting of (²H₆)dimethyl fumaric acid ester, (²H₃)methyl fumaric acid ester, (²H₃)dimethyl fumaric acid ester, dimethyl fumaric(2,3-²H₂) acid ester, methyl fumaric(2,3-²H₂) acid ester, ethyl fumaric(2,3-²H₂) acid ester, (²H₃)methyl fumaric(2,3-²H₂) acid ester, (²H₆)dimethyl fumaric(2,3-²H₂) acid ester, methyl (2-morpholino-2-oxoethyl)fumaric(2,3-²H₂) acid ester, methyl (4-morpholino-1-butyl) fumaric(2,3-²H²) acid ester, 2-(benzoyloxy)ethyl methyl fumaric(2,3-²H₂) acid ester, 2-(benzoyloxy)ethyl (²H₃)methyl fumaric acid ester, (S)-2-((2-amino-3-phenylpropanoyl)oxy)ethyl methyl fumaric(2,3-²H₂) acid ester, (S)-2-((2-amino-3-phenylpropanoyl)oxy)ethyl (²H₃)methyl fumaric acid ester, and pharmaceutically acceptable salts thereof.

In certain embodiments, the compound of Formula (V) is selected from 2-(benzoyloxy)ethyl methyl fumaric acid ester:

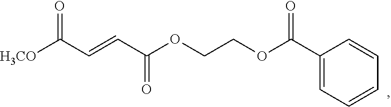 [SMILES: COC(=O)/C=C/C(=O)OCCOC(=O)c1ccccc1]

a deuterated analog thereof, or a pharmaceutically acceptable salt thereof.

In certain embodiments, the compound of Formula (V) is selected from (S)-2-((2-amino-2-phenylpropanoyl)oxy)ethyl methyl fumaric acid ester:

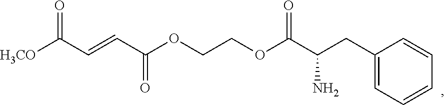 [SMILES: COC(=O)/C=C/C(=O)OCCOC(=O)[C@@H](N)Cc1ccccc1]

a deuterated analog thereof, or a pharmaceutically acceptable salt thereof.

In those embodiments where the compound of Formula (V) is a deuterated compound, the compound will have slightly altered and slower metabolism as compared with compounds of similar structure but lacking deuterium substitution.

In those embodiments where the compound of Formula (V) is a deuterated compound, the compound will have longer duration of action, increased exposure, and/or improved side effect profile as compared with compounds of similar structure but lacking deuterium substitution.

Certain embodiments of the methods disclosed herein utilize a compound of Formula (VI), as described Zeidan et al., U.S. Pat. No. 8,669,281, at column 7, line 24 through column 9, line 22, and column 15, line 55 to column 18, line 50, the disclosure of which is incorporated herein by reference. The compound of Formula (VI) include:

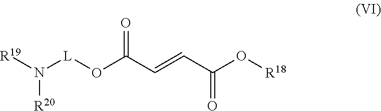

or a pharmaceutically acceptable salt thereof, wherein:

R¹⁸ is unsubstituted C₁-C₆ alkanyl;

L is substituted or unsubstituted C₁-C₆ alkanyl linker, substituted or unsubstituted C₃-C₁₀ cycloalkyl, substituted or unsubstituted C₆-C₁₀ aryl, substituted or unsubstituted cyclic heteroalkyl comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S; and

R¹⁹ and R²⁰ are each, independently, H, substituted or unsubstituted C₁-C₆ alkanyl, substituted or unsubstituted C₂-C₆ alkenyl, substituted or unsubstituted C₂-C₆ alkynyl, substituted or unsubstituted C₆-C₁₀ aryl, substituted or unsubstituted C₃-C₁₀ cycloalkyl, substituted or unsubstituted cyclic heteroalkyl comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S;

or alternatively, R¹⁹ and R²⁰, together with the nitrogen atom to which they are attached, form a substituted or unsubstituted heteroaryl comprising one or two 5- or 6-member rings and 1-4 heteroatoms selected from N, O and S or a substituted or unsubstituted cyclic heteroalkyl comprising one or two 5- or 6-member rings and 1-4 heteroatoms selected from N, O and S.

In certain embodiments of a compound of Formula (VI), R¹⁸ is methyl.

In certain embodiments of a compound of Formula (VI), R¹⁸ is ethyl.

In certain embodiments of a compound of Formula (VI), L is substituted or unsubstituted C₁-C₆ alkanyl linker.

In certain embodiments of a compound of Formula (VI), L is substituted or unsubstituted C₁-C₃ alkanyl linker.

In certain embodiments of a compound of Formula (VI), L is substituted or unsubstituted C₂ alkanyl linker.

In certain embodiments of a compound of Formula (VI), L is methyl substituted or unsubstituted C₂ alkanyl linker.

In certain embodiments of a compound of Formula (VI), L is di-methyl substituted or unsubstituted C₂ alkanyl linker.

In certain embodiments of a compound of Formula (VI), L is methyl or di-methyl substituted C₂ alkanyl linker.

In certain embodiments of a compound of Formula (VI), L is unsubstituted C₂ alkyl linker. In certain embodiments of a compound of Formula (VI),

R¹⁹ is substituted or unsubstituted C₁-C₆ alkanyl.

In certain embodiments of a compound of Formula (VI), R¹⁹ is unsubstituted C₁-C₆ alkanyl.

In certain embodiments of a compound of Formula (VI), R¹⁹ is unsubstituted C₁-C₃ alkanyl.

In certain embodiments of a compound of Formula (VI), R¹⁹ is unsubstituted C₁-C₂ alkanyl.

In certain embodiments of a compound of Formula (VI), R¹⁹ is C(O)OR²¹-substituted C₁-C₆ alkanyl, wherein R²¹ is H or unsubstituted C₁-C₆ alkanyl.

In certain embodiments of a compound of Formula (VI), R¹⁹ is S(O)(O)R²²-substituted C₁-C₆ alkanyl, wherein R²² is unsubstituted C₁-C₆ alkanyl.

In certain embodiments of a compound of Formula (VI), R²⁰ is H.

In certain embodiments of a compound of Formula (VI), R²⁰ is substituted or unsubstituted C₁-C₆ alkanyl.

In certain embodiments of a compound of Formula (VI), R²⁰ is unsubstituted C₁-C₆ alkanyl.

In certain embodiments of a compound of Formula (VI), R¹⁹ and R²⁰, together with the nitrogen atom to which they are attached, form a substituted or unsubstituted heteroaryl comprising one or two 5- or 6-member rings and 1-4 heteroatoms selected from N, O and S, or a substituted or unsubstituted cyclic heteroalkyl comprising one or two 5- or 6-member rings and 1-4 heteroatoms selected from N, O and S.

In certain embodiments of a compound of Formula (VI), R¹⁹ and R²⁰, together with the nitrogen atom to which they are attached, form a substituted or unsubstituted cyclic heteroalkyl comprising one or two 5- or 6-member rings and 1-4 heteroatoms selected from N, O and S.

In certain embodiments of a compound of Formula (VI), R¹⁹ and R²⁰, together with the nitrogen atom to which they are attached, form a substituted or unsubstituted pyrrolidinyl, imidazolidinyl, pyrazolidinyl, oxazolidinyl, isoxazolidinyl, triazolidinyl, tetrahydrofuranyl, piperidinyl, piperazinyl, or morpholinyl ring.

In certain embodiments of a compound of Formula (VI), R¹⁹ and R²⁰, together with the nitrogen atom to which they are attached, form a substituted or unsubstituted piperidinyl ring.

In certain embodiments of a compound of Formula (VI), R¹⁹ and R²⁰, together with the nitrogen atom to which they are attached, form an unsubstituted piperidinyl ring.

In certain embodiments of a compound of Formula (VI), R¹⁹ and R²⁰, together with the nitrogen atom to which they are attached, form a halogen substituted piperidinyl ring.

In certain embodiments of a compound of Formula (VI), R¹⁹ and R²⁰, together with the nitrogen atom to which they are attached, form a 4-halogen substituted piperidinyl ring.

In certain embodiments of a compound of Formula (VI), R¹⁹ and R²⁰, together with the nitrogen atom to which they are attached, form an unsubstituted morpholinyl ring.

In certain embodiments of a compound of Formula (VI), R¹⁹ and R²⁰, together with the nitrogen atom to which they are attached, form an unsubstituted pyrrolidinyl ring.

In certain embodiments of a compound of Formula (VI), R¹⁹ and R²⁰, together with the nitrogen atom to which they are attached, form a substituted or unsubstituted heteroaryl comprising one or two 5 or 6-member rings and 1-4 heteroatoms selected from N, O and S.

In certain embodiments of a compound of Formula (VI), R¹⁹ is substituted or unsubstituted C₆-C₁₀ aryl.

In certain embodiments of a compound of Formula (VI), R¹⁹ is unsubstituted C₆-C₁₀ aryl.

In certain embodiments of a compound of Formula (VI), R¹⁹ is unsubstituted phenyl.

In certain embodiments of a compound of Formula (VI), R¹⁹ is unsubstituted benzyl.

In certain embodiments, the compound of Formula (VI) is selected from the group consisting of

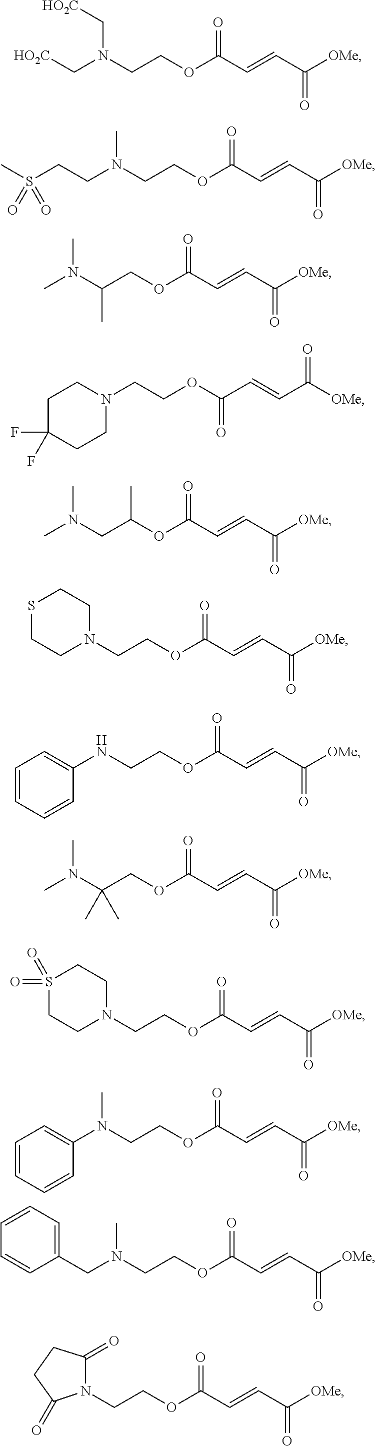

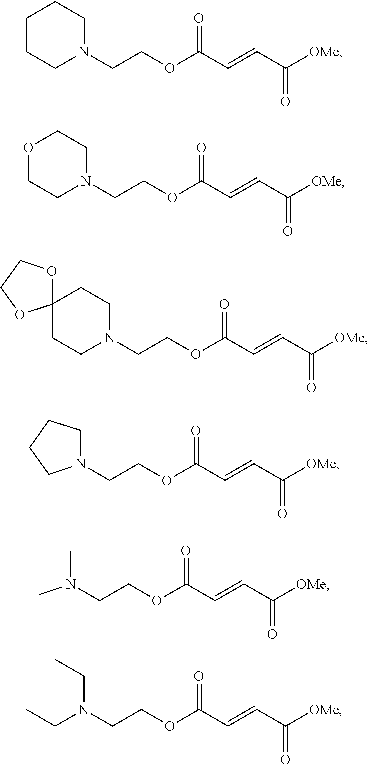 [SMILES: COC(=O)/C=C/C(=O)OCCN1CCCCC1 ; COC(=O)/C=C/C(=O)OCCN1CCOCC1 ; COC(=O)/C=C/C(=O)OCCN1CCC2(CC1)OCCO2 ; COC(=O)/C=C/C(=O)OCCN1CCCC1 ; COC(=O)/C=C/C(=O)OCCN(C)C ; CCN(CC)CCOC(=O)/C=C/C(=O)OC]

and pharmaceutically acceptable salts thereof.

In certain embodiments, the compound of Formula (VI) is selected from 2-(2,5-dioxopyrrolidin-1-yl)ethyl methyl fumarate:

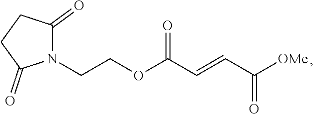 [SMILES: COC(=O)/C=C/C(=O)OCCN1C(=O)CCC1=O]

or a pharmaceutically acceptable salt thereof.

### Synthesis of Compounds

MMF can be synthesized according to the methods described in Dymicky, Preparation of Monomethyl Fumarate, Organic Preparations and Procedures International: The New Journal for Organic Synthesis, Vol 14, Issue 4, 1983; and Spatz et al., J. Org. Chem., 1958, 23 (10), 1559-1560, the disclosure of which is incorporated herein by reference.

DMF can be synthesized according to the methods described in Chinese Patent Publication CN 101318901A, the disclosure of which is incorporated herein by reference.

Compounds of Formula (I) can be synthesized according to the methods described in Speiser et al., U.S. Pat. No. 5,424,332, at column 3, line 33 through column 4, line 2, the disclosure of which is incorporated herein by reference.

Compounds of Formula (II) can be synthesized according to the methods described in Gangakhedkar et al., U.S. Pat. No. 8,148,414, at column 23, line 44 through column 26, line 55 and column 28, line 10 through column 29, line 34, the disclosure of which is incorporated herein by reference.

Compounds of Formula (III) can be synthesized according to the methods described in Gangakhedkar et al., U.S. Pat. No. 8,148,414, at column 29, line 43 through column 31, line 13, the disclosure of which is incorporated herein by reference.

Compounds of Formula (IV) can be synthesized according to the methods described in Cundy et al., U.S. patent application Ser. No. 13/761,864, filed Feb. 7, 2013, at page 34, line 21 through page 41, line 3, the disclosure of which is incorporated herein by reference.

Compounds of Formula (V) can be synthesized according to the methods described in Chao, US Patent Application Publication No. 2014/0194427 at paragraphs 54-37 and 113-150, the disclosure of which is incorporated herein by reference.

Compounds of Formula (VI) can be synthesized according to the methods described in Zeidan et al., U.S. Pat. No. 8,669,281, at column 27, line 45 through column 34, line 19, the disclosure of which is incorporated herein by reference.

### Pharmaceutical Compositions

Pharmaceutical compositions provided by the present disclosure may comprise a therapeutically effective amount of MMF and/or a prodrug of MMF together with a suitable amount of one or more pharmaceutically acceptable vehicles so as to provide a composition for proper administration to a patient. Suitable pharmaceutical vehicles are described in the art.

In certain embodiments, MMF and/or a compound of Formulae (I)-(VI) may be incorporated into pharmaceutical compositions to be administered orally. Oral administration of such pharmaceutical compositions may result in uptake of MMF and/or a compound of Formulae (I)-(VI) throughout the intestine and entry into the systemic circulation. Such oral compositions may be prepared in a manner known in the pharmaceutical art and comprise MMF and/or a compound of Formulae (I)-(VI) and at least one pharmaceutically acceptable vehicle. Oral pharmaceutical compositions may include a therapeutically effective amount of MMF and/or a compound of Formulae (I)-(VI) and a suitable amount of a pharmaceutically acceptable vehicle, so as to provide an appropriate form for administration to a patient.

MMF and/or a compound of Formulae (I)-(VI) may be incorporated into pharmaceutical compositions to be administered by any other appropriate route of systemic administration including intramuscular, intravenous and oral.

Pharmaceutical compositions comprising MMF and/or a compound of Formulae (I)-(VI) and may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of MMF and/or a compound of Formulae (I)-(VI) or crystalline forms thereof and one or more pharmaceutically acceptable vehicles into formulations that can be used pharmaceutically. Proper formulation is dependent upon the route of administration chosen. Pharmaceutical compositions provided by the present disclosure take the form of sustained-release formulations suitable for administration to a patient.

Pharmaceutical compositions provided by the present disclosure may be formulated in a unit dosage form. A unit dosage form refers to a physically discrete unit suitable as a unitary dose for patients undergoing treatment, with each unit containing a predetermined quantity of MMF and/or a compound of Formulae (I)-(VI) calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

In certain embodiments, an oral dosage form provided by the present disclosure may be a controlled release dosage form. Controlled delivery technologies can improve the absorption of a drug in a particular region or regions of the gastrointestinal tract. Controlled drug delivery systems may be designed to deliver a drug in such a way that the drug level is maintained within a therapeutically effective window, is effective and safe blood levels are maintained for a period as long as the system continues to deliver the drug with a particular release profile in the gastrointestinal tract. Controlled drug delivery may produce substantially constant blood levels of a drug over a period of time as compared to fluctuations observed with immediate release dosage forms. For some drugs, maintaining a constant blood and tissue concentration throughout the course of therapy is the most desirable mode of treatment. Immediate release of drugs may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

An appropriate oral dosage form for a particular pharmaceutical composition provided by the present disclosure may depend, at least in part, on the gastrointestinal absorption properties of MMF and/or a compound of Formulae (I)-(VI) the stability of MMF and/or a compound of Formulae (I)-(VI) in the gastrointestinal tract, the pharmacokinetics of MMF and/or a compound of Formulae (I)-(VI) and the intended therapeutic profile. An appropriate controlled release oral dosage form may be selected for a particular compound. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Certain compounds are absorbed primarily from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

In certain embodiments, pharmaceutical compositions provided by the present disclosure may be practiced with dosage forms adapted to provide sustained release of MMF and/or a compound of Formulae (I)-(VI) upon oral administration. Sustained release oral dosage forms may be used to release drugs over a prolonged time period and are useful when it is desired that a drug or drug form be delivered to the lower gastrointestinal tract, including the colon. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In another embodiment, the prodrug of MMF is a compound of Formulae (I)-(VI).

In certain embodiments, pharmaceutical compositions provided by the present disclosure may include any enteric-coated sustained release oral dosage form of MMF and/or a prodrug of MMF.

In one embodiment, the prodrug of MMF is a compound of Formulae (I)-(VI). In another embodiment, the enteric-coated oral dosage form is administered to a patient at a dosing frequency of not more than twice per day.

In certain embodiments, pharmaceutical compositions provided by the present disclosure may include any non-enteric-coated sustained release oral dosage form of MMF and/or a prodrug of MMF.

In one embodiment, the prodrug of MMF is a compound of Formulae (I)-(VI). In another embodiment, the non-enteric-coated oral dosage form is administered to a patient at a dosing frequency of not more than twice per day.

In certain embodiments, pharmaceutical compositions provided by the present disclosure may include any suitable dosage forms that achieve the above described in vitro release profiles. Such dosage forms may be any systemic dosage forms, including sustained release enteric-coated oral dosage form and sustained release non-enteric-coated oral dosage form. Examples of suitable dosage forms are described herein. Those skilled in the formulation art can develop any number of acceptable dosage forms given the dosage forms described in the examples as a starting point.

An appropriate dose of MMF and/or a compound of Formulae (I)-(VI) or pharmaceutical composition comprising MMF and/or a compound of Formulae (I)-(VI) may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

Compounds of Formulae (I)-(VI) are prodrugs of MMF. Thus, compounds of Formulae (I)-(VI) and pharmaceutical compositions thereof may be administered to a patient suffering from diseases, disorders, conditions, and symptoms of any of the foregoing for which alkyl hydrogen fumarates, such as MMF, are known to provide, or are later found to provide, therapeutic benefit. MMF and/or a compound of Formulae (I)-(VI) can be used to treat a disease chosen from adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis, Zellweger's syndrome, granulomas including annulaire, pemphigus, bollus pemphigoid, Behcet's, contact dermatitis, acute dermatitis, chronic dermatitis, alopecia greata (totalis and universalis), sarcoidosis, cutaneous sarcoidosis, pyoderma gangrenosum, cutaneous lupus, Crohn's disease or cutaneous Crohn's disease.

In other embodiments, MMF and/or a compound of Formulae (I)-(VI) can be used to treat a disease chosen from adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis, Zellweger's syndrome, granulomas including annulaire, pemphigus, bollus pemphigoid, Behcet's, contact dermatitis, acute dermatitis, chronic dermatitis, alopecia greata (totalis and universalis), sarcoidosis, cutaneous sarcoidosis, pyoderma gangrenosum, cutaneous lupus, Crohn's disease or cutaneous Crohn's disease.

In certain embodiments, MMF and/or a compound of Formulae (I)-(VI) can be used to treat a disease chosen from rheumatica, granuloma annulare, lupus, autoimmune carditis, eczema, sarcoidosis, acute disseminated encephalomyelitis, Addison's disease, alopecia greata, ankylosing spondylitis, antiphospholipid antibody syndrome, autoimmune hemolytic anemia, autoimmune hepatitis, autoimmune inner ear disease, bullous pemphigoid, Behcet's disease, celiac disease, Chagas disease, chronic obstructive pulmonary disease, Crohn's disease, dermatomyositis, diabetes mellitus type I, endometriosis, Goodpasture's syndrome, Graves' disease, Guillain-Barre syndrome, Hashimoto's disease, hidradenitis suppurativea, Kawasaki disease, IgA neuropathy, idiopathic thrombocytopenic purpura, interstitial cystitis, lupus erythematosus, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, schizophrena, scleroderma, Sjogren's syndrome, stiff person syndrome, temporal arteritis, ulcerative colitis, vasculitis, vitiligo, or Wegener's granulomatosis.

In certain embodiments, MMF and/or a compound of Formulae (I)-(VI) can be used to treat a disease chosen from psoriasis, asthma, chronic obstructive pulmonary disease, cardiac insufficiency, left ventricular insufficiency, myocardial infarction, angina pectoris, Parkinson's disease, Alzheimer's disease, Huntington's disease, retinopathia pigmentosa, mitochondrial encephalomyopathy, transplantation rejection, multiple sclerosis, ischemia, reperfusion injury, AGE-induced genome damage, inflammatory bowel disease, Crohn's disease, or ulcerative colitis.

In certain embodiments, MMF and/or a compound of Formulae (I)-(VI) can be used to treat a disease chosen from multiple sclerosis, psoriasis, alopecia greata, irritable bowel disorder, ulcerative colitis, arthritis, chronic obstructive pulmonary disease, asthma, Parkinson's disease, Huntington's disease, and amyotrophic lateral sclerosis. In certain embodiments, MMF and/or a compound of Formulae (I)-(VI) can be used to treat a disease chosen from psoriasis, multiple sclerosis, an inflammatory bowel disease, asthma, chronic obstructive pulmonary disease, or arthritis.

In certain embodiments, MMF and/or a compound of Formulae (I)-(VI) can be used to treat a disease chosen from multiple sclerosis, psoriasis, and alopecia greata. In certain embodiments, MMF and/or a compound of Formulae (I)-(VI) can be used to treat a disease chosen from multiple sclerosis and psoriasis. In certain embodiments, MMF and/or a compound of Formulae (I)-(VI) can be used to treat a disease chosen from multiple sclerosis and alopecia greata. In certain embodiments, MMF and/or a compound of Formulae (I)-(VI) can be used to treat a disease chosen from psoriasis and alopecia greata. In certain embodiments, other embodiments, MMF and/or a compound of Formulae (I)-(VI) can be used to treat multiple sclerosis. In certain embodiments, MMF and/or a compound of Formulae (I)-(VI) can be used to treat psoriasis. In certain embodiments, MMF and/or a compound of Formulae (I)-(VI) can be used to treat alopecia greata. In certain embodiments, the alopecia greata is alopecia greata totalis. In certain embodiments, the alopecia greata is alopecia greata universalis.

As generally recognized by those in the art, “AGE-induced genome damage” refers to genomic damage caused by advanced glycation endproducts (AGEs), which are non-enzymatic glycation products of proteins and lipids leading to reduced kidney function and an increased occurrence of diabetes mellitus. See WO 2005/027899, supra, especially at p. 2, ll. 8-17, which is incorporated by reference in its entirety.

Methods of treating a disease in a patient provided by the present disclosure comprise administering to a patient in need of such treatment a therapeutically effective amount of MMF and/or a compound of Formulae (I)-(VI). These compounds, and pharmaceutical compositions thereof, provide therapeutic or prophylactic plasma and/or blood concentrations of MMF following administration to a patient. MMF and/or a compound of Formulae (I)-(VI) may be administered in an amount and using a dosing schedule as appropriate for treatment of a particular disease. Daily doses of MMF and/or a compound of Formulae (I)-(VI) may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. In certain embodiments, MMF and/or a compound of Formulae (I)-(VI) may be administered at a dose over time from about 1 mg to about 5 g per day, from about 10 mg to about 4 g per day, and in certain embodiments from about 20 mg to about 2 g per day. An appropriate dose of MMF and/or a compound of Formulae (I)-(VI) may be determined based on several factors, including, for example, the bodyweight and/or condition of the patient being treated, the severity of the disease being treated, the incidence and/or severity of side effects, the manner of administration, and the judgment of the prescribing physician. Appropriate dose ranges may be determined by methods known to those skilled in the art.

MMF and the compounds of Formulae (I)-(VI) may be assayed in vitro and in vivo for the desired therapeutic or prophylactic activity prior to use in humans. In vivo assays, for example using appropriate animal models, may also be used to determine whether administration of MMF and/or a compound of Formulae (I)-(VI) is therapeutically effective.

In certain embodiments, a therapeutically effective dose of MMF and/or a compound of Formulae (I)-(VI) may provide therapeutic benefit without causing substantial toxicity including adverse side effects. Toxicity of MMF and/or a compound of Formulae (I)-(VI) and/or metabolites thereof may be determined using standard pharmaceutical procedures and may be ascertained by those skilled in the art. The dose ratio between toxic and therapeutic effect is the therapeutic index. A dose of MMF and/or a compound of Formulae (I)-(VI) may be within a range capable of establishing and maintaining a therapeutically effective circulating plasma and/or blood concentration of MMF and/or a compound of Formulae (I)-(VI) that exhibits little or no toxicity.

MMF and compounds of Formulae (I)-(VI) may be used to treat a disease chosen from adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis, Zellweger's syndrome, granulomas including annulaire, pemphigus, bollus pemphigoid, Behcet's, contact dermatitis, acute dermatitis, chronic dermatitis, alopecia greata (totalis and universalis), sarcoidosis, cutaneous sarcoidosis, pyoderma gangrenosum, cutaneous lupus, Crohn's disease or cutaneous Crohn's disease. The underlying etiology of any of the foregoing diseases being treated may have a multiplicity of origins. Further, in certain embodiments, a therapeutically effective amount of MMF and/or the compound of Formulae (I)-(VI) may be administered to a patient, such as a human, as a preventative measure against the foregoing diseases and disorders. Thus, a therapeutically effective amount of MMF and/or a compound of Formulae (I)-(VI) may be administered as a preventative measure to a patient having a predisposition for and/or history of adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis, Zellweger's syndrome, granulomas including annulaire, pemphigus, bollus pemphigoid, Behcet's disease, contact dermatitis, acute dermatitis, chronic dermatitis, alopecia greata (totalis and universalis), sarcoidosis, cutaneous sarcoidosis, pyoderma gangrenosum, cutaneous lupus, Crohn's disease and/or cutaneous Crohn's disease.

### Administration

MMF and/or a prodrug of MMF and pharmaceutical compositions thereof may be administered orally or by any other appropriate route suitable for systemic, as opposed to local, administration. For example, systemic administration can be by infusion or bolus injection, by absorption through epithelial or mucocutaneous linings (e.g., oral mucosa, rectal, and intestinal mucosa, etc.). Other suitable routes of systemic administration include, but are not limited to, intradermal, intramuscular, intraperitoneal, intravenous, subcutaneous, intranasal, epidural, oral, sublingual and inhalation.

The amount of MMF and/or a prodrug of MMF that will be effective in the treatment of a disease in a patient will depend, in part, on the nature of the condition and can be determined by standard clinical techniques known in the art. In addition, in vitro or in vivo assays may be employed to help identify optimal dosage ranges. A therapeutically effective amount of MMF and/or a prodrug of MMF to be administered may also depend on, among other factors, the subject being treated, the weight of the subject, the severity of the disease, the manner of administration, and the judgment of the prescribing physician. In the case of an MMF prodrug, for which MMF is the pharmacologically active metabolite, the amount of prodrug to be administered is generally determined by calculating the weight of any pharmacologically inactive promoiety that is cleaved during metabolism of the prodrug and then administering a MMF equivalent amount of the prodrug.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

A dose may be administered in a single dosage form or in multiple dosage forms. When multiple dosage forms are used the amount of compound contained within each dosage form may be the same or different. The amount of MMF and/or a prodrug of MMF contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, MMF and/or a prodrug of MMF may exhibit a high therapeutic index. The data obtained from these cell culture assays and animal studies may be used in formulating a dosage range that is not toxic for use in humans. A dose of MMF and/or a prodrug of MMF provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Combination Therapy

Methods provided by the present disclosure further comprise administering one or more pharmaceutically active compounds in addition to MMF and/or a prodrug of MMF. Such compounds may be provided to treat the same disease or a different disease than the disease being treated with the MMF and/or MMF prodrug.

In certain embodiments, MMF and/or an MMF prodrug may be used in combination with at least one other therapeutic agent. In certain embodiments, MMF and/or a MMF prodrug may be administered to a patient together with another compound for treating diseases and conditions including: adrenal leukodystrophy, Alexanders Disease, Alper's Disease, balo concentric sclerosis, Canavan disease, central nervous system vasculitis, Charcott-Marie-Tooth Disease, childhood ataxia with central nervous system hypomyelination, diabetic retinopathy, graft versus host disease, hepatitis C viral infection, herpes simplex viral infection, human immunodeficiency viral infection, Krabbe Disease, lichen planus, macular degeneration, monomelic amyotrophy, neurodegeneration with brain iron accumulation, neuromyelitis optica, neurosarcoidosis, optic neuritis, pareneoplastic syndromes, Pelizaeus-Merzbacher disease, primary lateral sclerosis, progressive supranuclear palsy, Schilders Disease, subacute necrotizing myelopathy, susac syndrome, transverse myelitis, a tumor and Zellweger's syndrome.

MMF and/or an MMF prodrug and the additional other therapeutic agent may act additively or, and in certain embodiments, synergistically. The additional other therapeutic agent may be included in the same dosage form as MMF and/or the MMF prodrug or may be provided in a separate dosage form. Methods provided by the present disclosure can further include, in addition to administering MMF and/or an MMF prodrug, administering one or more therapeutic agents effective for treating the same or different disease than the disease being treated by MMF and/or the MMF prodrug. Methods provided by the present disclosure include administration of MMF and/or an MMF prodrug and one or more other therapeutic agents provided that the combined administration does not inhibit the therapeutic efficacy of the MMF and/or the MMF prodrug and/or does not typically produce significant and/or substantial adverse combination effects.

In certain embodiments, dosage forms comprising MMF and/or a prodrug of MMF may be administered concurrently with the administration of another therapeutic agent, which may be part of the same dosage form as, or in a different dosage form than that comprising MMF and/or a prodrug of MMF. MMF and/or a prodrug of MMF may be administered prior or subsequent to administration of another therapeutic agent. In certain embodiments of combination therapy, the combination therapy may comprise alternating between administering MMF and/or a prodrug of MMF and a composition comprising another therapeutic agent, e.g., to minimize adverse drug effects associated with a particular drug. When MMF and/or a prodrug of MMF is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising MMF and/or a prodrug of MMF may be administered with one or more substances to enhance, modulate and/or control release, bioavailability, therapeutic efficacy, therapeutic potency, stability, and the like of MMF and/or a prodrug of MMF. For example, to enhance the therapeutic efficacy of a MMF and/or a prodrug of MMF, the MMF and/or a prodrug of MMF may be co-administered with or a dosage form comprising MMF and/or a prodrug of MMF may comprise one or more active agents to increase the absorption or diffusion of MMF and/or a prodrug of MMF from the gastrointestinal tract to the systemic circulation, or to inhibit degradation of the MMF and/or a prodrug of MMF in the blood of a patient. In certain embodiments, MMF and/or a prodrug of MMF may be co-administered with an active agent having pharmacological effects that enhance the therapeutic efficacy of a MMF and/or a prodrug of MMF.

### EXAMPLES

The following examples illustrate various aspects of the disclosure. It will be apparent to those skilled in the art that many modifications, both to materials and methods, may be practiced without departing from the scope of the disclosure.

### Example 1

### Testing for Genotypes GSTT1*A/A, GSTT1*A/0 and GSTT1*0/0

For initial determination and characterization of the GSTT1 deletion, samples of Caucasian volunteers from the Dr. Margarete Fischer-Bosch-Institute of Clinical Pharmacology in Stuttgart, and from the Institute of Clinical Pharmacology at the University Medical Center, Charite in Berlin have been used. Samples were obtained under consideration of all ethical and legal requirements. Genomic DNA was prepared from blood using the Qiagen (QiaAmp) kits on a Qiagen 9604 robot. For geno-phenotype correlations, phenotyped subjects have been used (n=130, male, mean age 30.7 years, ranging from 22 to 49 years) which were part of a previous study (Bruhn et al. 1998), DNA was obtained from these samples using phenol/chloroform extraction.

Determination of formaldehyde production rate (pmol HCHO/min/.mu.l) from 31, 62, and 124 mM dichloromethane in hemolysate was used as a measure for GSTT1 activity following the methods described by Bruhn et al. (Bruhn et al., Concordance between enzyme activity and genotype of glutathione S-transferase theta (GSTT1), Biochem Pharmacol; 56:1189-1193, 1998).

NCBI database entries Z84718.1 and AP000351.2 (Genbank) contain GSTT1 sequences in annotated form (Z84718.1) or as raw data files, respectively. DNA sequence comparisons, alignments and the construction of composite files from raw data sequence files were performed using the programs FASTA and BLAST at the NCBI server as described in Altschul et al. (Altschul et al., Gapped BLAST and PSI-BLAST: a new generation of protein database search programs, Nucleic Acids Res; 25:3389-3402, 1997). A composite sequence of the GSTT1 gene region is deposited at Genbank: AF240786. The sequence of the deletion allele is deposited at Genbank: AF240785.

Specific oligonucleotide primers for PCR of GSTT1 gene fragments were derived from Genbank: AF240786 (Table 1). Sequences of purified PCR fragments were obtained by automated DNA sequencing on ABI 377 (gel) or ABI 3700 (capillary) sequencers using BigDye Terminator cycle sequencing reactions (PE Biosystems, Weiterstadt, Germany). Amplification of fragments less than 2 kb was performed in 25 μl volume: 100 ng DNA template added to buffer containing 1.5 mM MgCl₂, 200 μM dNTPs, 0.2 mM each primer and 1 U HotStarTaq polymerase (all reagents Qiagen, Hilden, Germany). PCR was carried out in a Perkin Elmer GeneAmp System 9700 with an initial denaturation of 15 min at 95° C. followed by 30 cycles of 94° C. for 30 s, 30 s annealing and 60 s of extension at 72° C. Final extension was carried out for 7 min at 72° C. For longer amplicons 50 μl PCR reactions contain 200 ng of genomic DNA, reaction buffer 3, 500 μM dNTPs, and 2.6 U Expand Taq-System (Roche, Basel, Switzerland) and 0.3 mM primers (Metabion, Munich, Germany). Samples were incubated at 92° C. for 2 min, followed by 35 cycles at 92° C. for 10 s, 45 s annealing at 68° C. for each kb per min extension time. The extension time of each cycle was increased by 20 s for the last 25 cycles. 10 min final extension at 68° C. was applied. PCR fragments were analysed on 1.5% agarose gels in TBE-buffer to separate fragments lower than 3 kb and 0.8% agarose gels for resolution of larger fragments. Gels were stained with ethidium bromide and the data documented digitally. For genotyping, the 1460 bp GSTT1*0 specific fragment was co-amplied with the 460 bp GSTT1*A fragment in a single reaction tube.

TABLE 1
Polymerase chain reaction (PCR) fragments for GSTT1 genotyping

| | | Size | | |
|---|---|---|---|---|
| Sequence | Position* | (bp) | Specificity | Comment |
| CTTTTTCTGCACCAAACGCATTG | 45986-46008 | 10065 | GSTT1*0 | Long range |
| | | | | PCR |
| GATGCCACGCGGCTTGTAGG | 110301-110282 | | | |
| ATATCAGCCAGAGATCTCTGGG | 50600-50621 | 3187 | GSTT1*0 | Long range |
| | | | | PCR |
| CAGCCAAGAAGTTCTGAGTCTTG | 108037-108015 | | | |
| CAGTTGTGAGCCACCGTACCC | 52069-52089 | 1460 | GSTT1*0 | Standard |
| | | | | PCR |
| CGATAGTTGCTGGCCCCCTC | 107779-107760 | | | |
| CCAGCTCACCGGATCATGGCCAG | 85457-85479 | 466 | GSTT1*A | Standard |
| | | | | PCR** |
| CCTTCCTTACTGGTCCTCACATCTC | 85922-85898 | | | |

*According to Genbank: AF240786.
**Modifed from Pemble et al., Human glutathione S-transferase theta (GSTT1): cDNA cloning and the characterization of a genetic polymorphism, Biochem J; 300: 271-276 (1994)

### Example 2

### Method of Determining GSTT1 Genotype

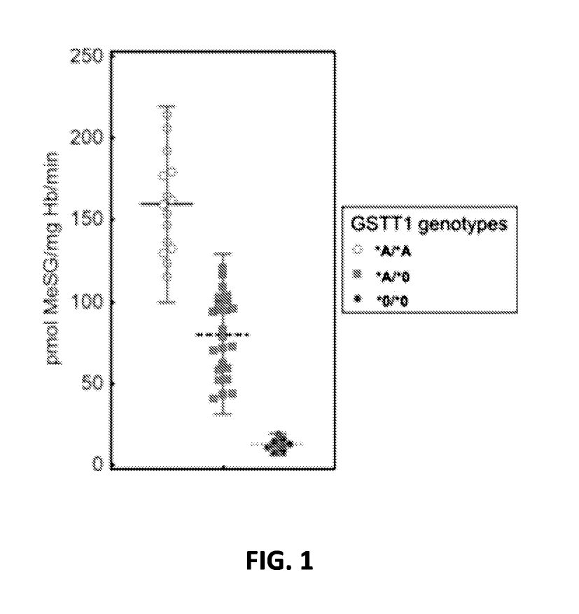

GSTT1 genotyping was performed in accordance with the method described by Sprenger et al. (Sprenger et al., Characterization of the glutathione S-transferase GSTT1 deletion: discrimination of all genotypes by polymerase chain reaction indicates a trimodular genotype-phenotype correlation. Pharmacogenetics 2000; 10: 557-565) who found in 130 German subjects 34% homozygous (*A/*A), 46% heterozygous (*A/*0) and 20% homozygous deleted (*0/*0) GSTT1 gene. For GSTT1 genotyping blood samples of 48 psoriasis patients were available. Specific oligonucleotide primers for polymerase chain reaction (PCR) of GSTT1 gene fragments were derived from Genbank: AF240786 and AF240785. PCR fragments were analysed on 1.5% agarose gels in TAE-buffer. Gels were stained with ethidium bromide and the data documented digitally. For genotyping, the 1460 bp GSTT1*0 specific fragment was co-amplified with the 460 bp GSTT1*A fragment in a single reaction tube following the methods used by Sprenger et al. (supra). Based on GSTT1 genotyping, the GSTT1 conjugator classes were defined as follows: non-conjugators (NC), <25 pmol MeSG/mg Hb/min; low-conjugators (LC), 25 to 110 MeSG/mg Hb/min; and high-conjugators (HC), >110 MeSG/mg Hb/min. The results are shown in FIG. 1.

### Example 3

### Method of Determining GSTT1 Phenotype

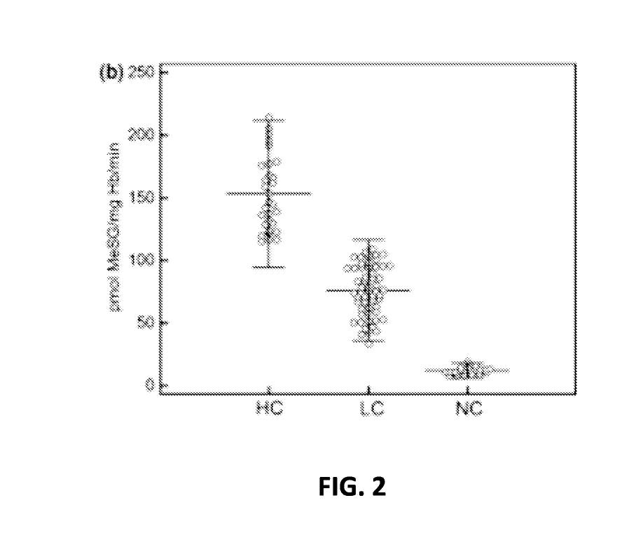

For long-term storage, erythrocyte lysates were prepared immediately after collection of the EDTA blood samples. Aliquots of the lysates were frozen and stored at −70° C. Methyl chloride (MeCl) was purchased from Messer Griesheim (Krefeld, Germany). High performance liquid chromatography (HPLC) ultra gradient acetonitrile was a product of Merck (Darmstadt, Germany). Water was purified by passage through an Elix 3 and Milli-Q system (Millipore, Eschborn, Germany). This water was used for all aqueous solutions and buffers. Using the HPLC procedure published by Muller et al. (Muller M, High-performance liquid chromatography/fluorescence detection of S-methylglutathione formed by glutathione-S-transferase T1 in vitro, Arch Toxicol 2001; 74: 760-767), 1 mL erythrocyte lysate corresponds to 1 mL initial EDTA blood. For the phenotyping of each individual, three incubations were carried out and analysed for S-methylglutathione (MeSG) formation using the methods disclosed by Muller et al. (supra). Two samples were exposed to 10,000 ppm MeCl (240 ll) and the third sample served as a control (no substrate added). Reproducibility data and the limits of quantitation and detection were as reported earlier. MeSG formation rates of the two MeCl-exposed samples of each individual were calculated as nanomoles MeSG/ml erythrocyte lysate per min and were averaged. To further standardize the expression of the hGSTT1-1 activity, the Hb value served as a surrogate for the enzyme protein content in the erythrocytes. Thus, each averaged MeSG formation rate was related to the Hb value determined in 1 mL EDTA blood (corresponding to 1 mL lysate) and the individual hGSTT1-1 activity was expressed as picomoles S-methylglutathione formed per mg hemoglobin per minute (pmol MeSG/mg Hb/min). The results are shown in FIG. 2.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

Having described several embodiments, it will be recognized by those skilled in the art that various modifications, alternative constructions, and equivalents may be used without departing from the spirit of the disclosure. In particular, any description of any R-group or chemical substituent, alone or in any combination, may be used in any chemical Formula described herein, and Formulae include all conformational and stereoisomers, including diastereomers, epimers, and enantiomers. Disclosures of all publication described herein are incorporated by reference in their entireis for all purposes. Additionally, a number of well-known processes and elements have not been described in order to avoid unnecessarily obscuring the embodiments disclosed herein. Accordingly, the above description should not be taken as limiting the scope of the document.

Those skilled in the art will appreciate that the presently disclosed embodiments teach by way of example and not by limitation. Therefore, the matter contained in the above description or shown in the chemical Formulae should be interpreted as illustrative and not in a limiting sense. The following claims are intended to cover all generic and specific features described herein, as well as all statements of the scope of the present method and system, which, as a matter of language, might be said to fall there between.

1. A method of treating a disease in a patient in need of such treatment, comprising:
(a) testing the patient for a propensity for a deficiency in tissue glutathione S-transferase theta 1 enzyme (GSTT1) levels;
(b) thereafter, administering to the patient a therapeutically effective amount of a compound selected from monomethyl fumarate (MMF), a prodrug of monomethyl fumarate, and combinations thereof; and
(c) during the treatment of the disease, periodically testing blood lymphocyte concentration in the patient at a predetermined time interval length that is based on the enzyme level propensity testing result.

2. The method of claim 1, wherein the enzyme level propensity testing comprises determining whether the patient exhibits a GSTT1*0/0 genotype, a GSTT1*A/A genotype or a GSTT1*A/0 genotype.

3. The method of claim 1, wherein the enzyme level propensity testing comprises measuring the amount of S-methyl glutathione formed upon exposure of patient hemoglobin to methyl chloride.

4. The method of claim 1, wherein the enzyme level propensity testing comprises measuring the amount of S-methyl glutathione formed upon exposure of patient lymphocytes to methyl chloride.

5. The method of claim 1, wherein the predetermined time interval length is shorter if the enzyme testing shows a propensity for a deficiency in tissue GSTT1 levels and longer if the enzyme testing shows little or no propensity for a deficiency in patient tissue GSTT1 levels.

6. The method of claim 2, wherein the predetermined time interval length is shorter if the patient exhibits a GSTT1*0/0 genotype; and longer if the patient exhibits a GSTT1*A/A genotype or a GSTT1*A/0 genotype.

7. The method of claim 6, wherein the patient exhibits a GSTT1*0/0 genotype and the predetermined time interval length is from about 1 to 6 months.

8. The method of claim 6, wherein the patient exhibits a GSTT1*A/0 genotype and the predetermined time interval length is from about 2 to 8 months.

9. The method of claim 6, wherein the patient exhibits a GSTT1*A/A genotype and the predetermined time interval length is greater than about 6 months.

10. The method of claim 6, wherein the patient exhibits a GSTT1*A/A genotype and the predetermined time interval length is greater than about 9 months.

11. The method of claim 3, wherein the predetermined time interval length is shorter if the patient exhibits at least 110 pmol MeSG/mg Hb/min; and longer if the patient exhibits less than 110 pmol MeSG/mg Hb/min.

12. The method of claim 3, wherein the patient exhibits less than 25 pmol MeSG/mg Hb/min and the predetermined time interval length is from about 1 to 6 months.

13. The method of claim 3, wherein the patient exhibits from about 25 to 110 pmol MeSG/mg Hb/min and the predetermined time interval length is from about 2 to 8 months.

14. The method of claim 3, wherein the patient exhibits greater than 110 pmol MeSG/mg Hb/min and the predetermined time interval length is greater than about 6 months.

15. The method of claim 2, wherein the genotype testing comprises GSTT1 genotyping a blood or tissue sample of the patient using specific oligonucleotide primers for polymerase chain reaction (PCR) of GSTT1 gene fragments.

16. The method of claim 2, wherein the genotype testing comprises using specific oligonucleotide primers of GSTT1 RNA for reverse transcription followed by polymerase chain reaction (PCR).

17. The method of claim 1, wherein the treatment of the disease is suspended if the lymphocyte testing shows an abnormally low lymphocyte blood concentration.

18. The method of claim 17, wherein the patient is a child and the abnormally low lymphocyte blood concentration is a level below about 3000 cells/μl.

19. The method of claim 17, wherein the patient is an adult and the abnormally low lymphocyte blood concentration is a level below about 1500 cells/μl.

20. The method of claim 1, wherein the disease is selected from multiple sclerosis and psoriasis.

21. The method of claim 1, wherein the disease is selected from adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis, Zellweger's syndrome, granulomas including annulaire, pemphigus, bollus pemphigoid, Behcet's, contact dermatitis, acute dermatitis, chronic dermatitis, alopecia areata (totalis and universalis), sarcoidosis, cutaneous sarcoidosis, pyoderma gangrenosum, cutaneous lupus, Crohn's disease and cutaneous Crohn's disease.

22. The method of claim 1, wherein the administered compound comprises monomethyl fumarate.

23. The method of claim 1, wherein the administered compound comprises a prodrug of monomethyl fumarate.

24. A method of treating a disease in a population of patients in need of such treatment, comprising:
(a) testing each of the patients for a propensity for a deficiency in tissue glutathione S-transferase theta 1 enzyme (GSTT1) levels;
and thereafter performing one of (b) or (c);
(b) administering a compound selected from monomethyl fumarate (MMF), a prodrug of monomethyl fumarate, and combinations thereof only to those patients exhibiting little or no propensity for a deficiency in tissue GSTT1 levels; or
(c) administering a compound selected from monomethyl fumarate (MMF), a prodrug of monomethyl fumarate, and combinations thereof to the patients regardless of their enzyme level propensity testing results, and during the treatment of the disease, periodically testing blood lymphocyte concentrations in each of the patients at a predetermined time interval length that is based on each patient's own enzyme level propensity testing result.

25-47. (canceled)

48. A method of optimizing the safety of treatment of a compound selected from MMF, a prodrug of MMF and combinations thereof, in a patient in need of such treatment, comprising:
testing the patient for a propensity for a deficiency in tissue glutathione S-transferase theta 1 enzyme (GSTT1) levels; wherein, if said testing shows little or no propensity for a deficiency in tissue GSTT1 levels:
administering to the patient a therapeutically effective amount of the compound; and
periodically testing blood lymphocyte concentration in the patient at a predetermined time interval length that is based on the enzyme level propensity testing result.


SEQUENCE LISTING (8 sequences)

SEQ ID NO: 1 - DNA, 23 bp, Artificial Sequence
  Synthetic
ctttttctgcaccaaacgcattg

SEQ ID NO: 2 - DNA, 20 bp, Artificial Sequence
  Synthetic
gatgccacgcggcttgtagg

SEQ ID NO: 3 - DNA, 22 bp, Artificial Sequence
  Synthetic
atatcagccagagatctctggg

SEQ ID NO: 4 - DNA, 23 bp, Artificial Sequence
  Synthetic
cagccaagaagttctgagtcttg

SEQ ID NO: 5 - DNA, 21 bp, Artificial Sequence
  Synthetic
cagttgtgagccaccgtaccc

SEQ ID NO: 6 - DNA, 20 bp, Artificial Sequence
  Synthetic
cgatagttgctggccccctc

SEQ ID NO: 7 - DNA, 23 bp, Artificial Sequence
  Synthetic
ccagctcaccggatcatggccag

SEQ ID NO: 8 - DNA, 25 bp, Artificial Sequence
  Synthetic
ccttccttactggtcctcacatctc